SEQUENCE LISTING (45 sequences)

SEQ ID NO: 1 - protein, 724 aa, Adeno-associated virus 5
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 2 - protein, 42 aa, Adeno-associated virus 2
PVATEQYGSVSTNLQRGNRQAATADVNTQGVLPGMVWQDRDV

SEQ ID NO: 3 - protein, 42 aa, Adeno-associated virus 1
PVATERFGTVAVNFQSSSTDPATGDVHAMGALPGMVWQDRDV

SEQ ID NO: 4 - protein, 42 aa, Adeno-associated virus 5
RVAYNVGGQMATNNQSSTTAPATGTYNLQEIVPGSVWMERDV

SEQ ID NO: 5 - protein, 42 aa, Adeno-associated virus 6
PVATERFGTVAVNLQSSSTDPATGDVHVMGALPGMVWQDRDV

SEQ ID NO: 6 - protein, 42 aa, Adeno-associated virus 7
PVATEEYGIVSSNLQAANTAAQTQVVNNQGALPGMVWQNRDV

SEQ ID NO: 7 - protein, 42 aa, Adeno-associated virus 8
PVATEEYGIVADNLQQQNTAPQIGTVNSQGALPGMVWQNRDV

SEQ ID NO: 8 - protein, 42 aa, Adeno-associated virus 9
PVATESYGQVATNHQSAQAQAQTGWVQNQGILPGMVWQDRDV

SEQ ID NO: 9 - protein, 42 aa, Adeno-associated virus 10
PVATEQYGVVADNLQQANTGPIVGNVNSQGALPGMVWQNRDV

SEQ ID NO: 10 - protein, 7 aa, synthetic construct
KVTRGDT

SEQ ID NO: 11 - skipped

SEQ ID NO: 12 - protein, 7 aa, synthetic construct
VTNVVRA

SEQ ID NO: 13 - protein, 7 aa, synthetic construct
PGPGNTI

SEQ ID NO: 14 - protein, 7 aa, synthetic construct
QRIVNEV

SEQ ID NO: 15 - protein, 1368 aa, Streptococcus pyogenes [sequence omitted]

SEQ ID NO: 16 - protein, 7 aa, synthetic construct
RSSNYVV

SEQ ID NO: 17 - protein, 7 aa, synthetic construct
AAPIDGE

SEQ ID NO: 18 - protein, 7 aa, synthetic construct
IGPVAAD

SEQ ID NO: 19 - protein, 7 aa, synthetic construct
DGVEDAV

SEQ ID NO: 20 - protein, 7 aa, synthetic construct
GFTDDAT

SEQ ID NO: 21 - protein, 7 aa, synthetic construct
LEAVATV

SEQ ID NO: 22 - protein, 14 aa, synthetic construct
VEAVGGNVEAVGGN

SEQ ID NO: 23 - protein, 7 aa, synthetic construct
MSLLPYP

SEQ ID NO: 24 - protein, 7 aa, synthetic construct
YFAIYIF

SEQ ID NO: 25 - protein, 7 aa, synthetic construct
HLSDARP

SEQ ID NO: 26 - protein, 7 aa, synthetic construct
LIASFVQ

SEQ ID NO: 27 - protein, 7 aa, synthetic construct
AGGGGKA

SEQ ID NO: 28 - protein, 7 aa, synthetic construct
KGVQERA

SEQ ID NO: 29 - protein, 7 aa, synthetic construct
AVRINPG

SEQ ID NO: 30 - protein, 736 aa, Adeno-associated virus 9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 31 - DNA, 2636 bp, synthetic construct
  source 1..2636: mol_type = other DNA
aatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttgcgatttaaatcaggtatggctgccgatggttatcttccagattggctcgaggacaacctctctgagggcattcgcgagtggtgggcgctgaaacctggaaccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagagggttctcgaaccttttggtctggttgaggaaggtgctaagacggctcctggaaagaaacgtccggtagagcagtcgccacaagagccagactcctcctcgggcattggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtctttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagacgtgtaccttcaggggcccatctgggcaaagattccacacacggacggacattttcacccctctcccctcatgggtggattcggacttaaacaccctcctccccagattctcatcaagaacaccccggtacctgcgaatccttcgaccaccttcaaccagtcaaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaattgcggccgcttgttaatcaataaaccgtttaattcgtttcagttgaactttggtctctgcgtatttctttcttatctagtttccatgcctcgagataacttcgtataatgtatgctatacgaacggtactgt

SEQ ID NO: 32 - DNA, 2648 bp, synthetic construct
  source 1..2648: mol_type = other DNA
aacaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtctttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaattgcggccgcttgttaatcaataaaccgtttaattcgtttcagttgaactttggtctctgcgtatttctttcttatctagtttccatgcctcgagataacttcgtataatgtatgctatacgaacggtactgtggtcgtcatgcaa

SEQ ID NO: 33 - DNA, 2639 bp, synthetic construct
  source 1..2639: mol_type = other DNA
aacaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttgcgatttaaatcaggtatggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccggcaacggactcgacaagggggagcccgtcaacgcggcggacgcagcggccctcgagcacgacaaggcctacgaccagcagctcaaagcgggtgacaatccgtaccttcggtataaccacgccgacgccgagtttcaggagcgtctgcaagaagatacgtcttttgggggcaacctcggacgagcagtcttccaggccaagaaaagggttctcgaaccttttggtctggttgaggaagcggctaagacggctcctggaaagaaacgtccggtagagcagtcgccacaagagccagactcctcctcgggcatcggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcagacaataacgaaggcgccgacggagtgggtagttcctcgggaaattggcattgcgattccacatggctgggcgacagagtcatcaccaccagcacccgcacctgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaactggggattccggcccaagagactcaacttcaaactcttcaacatccaagtcaaggaggtcacgacgaatgatggcgtcacaaccatcgctaataaccttaccagcacggttcaagtcttctcggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaaccggcaacaacttccagtttacttacaccttcgaggacgtgcctttccacagcagctacgcgcacagccagagcctggaccggctgatgaatcctctcatcgaccagtacctgtattacctgaacagaactcagaatcagtccggaagtgcccaaaacaaggacttgctgtttagccgtgggtctccagctggcatgtctgttcagcccaaaaactggctacctggaccctgttatcggcagcagcgcgtttctaaaacaaaaacagacaacaacaacagcaattttacctggactggtgcttcaaaatataacctcaatgggcgtgaatccatcatcaaccctggcactgctatggcctcacacaaagacgacgaagacaagttctttcccatgagcggtgtcatgatttttggaaaagagagcgccggagcttcaaacactgcattggacaatgtcatgattacagacgaagaggaaattaaagccactaaccctgtggccaccgaaagatttgggaccgtggcagtcaatttccagagcagcagcacagaccctgcgaccggagatgtgcatgctatgggagcattacctggcatggtgtggcaagatagagacgtgtacctgcagggtcccatttgggccaaaattcctcacacagatggacactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttacctcacccgtcccctgtaattgcggccgcttgttaatcaataaaccgtttaattcgtttcagttgaactttggtctctgcgtatttctttcttatctagtttccatgcctcgagataacttcgtataatgtatgctatacgaacggtactgt

SEQ ID NO: 34 - protein, 736 aa, synthetic construct
MAADGYLPDWLEDNLSEGIREWWALKPGTPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFNQSKLNSFITQYSTGQVSVEIEWELQKENSKRWNPEIQYTSNYYKSNNVEFAVNTEGVYSEPRPIGTRYLTRNL

SEQ ID NO: 35 - protein, 737 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSNNVEFAVNTEGVYSEPRPIGTRYLTRNL

SEQ ID NO: 36 - protein, 736 aa, synthetic construct
MAADGYLPDWLEDNLSEGIREWWALKPGAPKPKANQQKQDDGRGLVLPGYKYLGPGNGLDKGEPVNAADAAALEHDKAYDQQLKAGDNPYLRYNHADAEFQERLQEDTSFGGNLGRAVFQAKKRVLEPFGLVEEAAKTAPGKKRPVEQSPQEPDSSSGIGKTGQQPAKKRLNFGQTGDSESVPDPQPLGEPPATPAAVGPTTMASGGGAPMADNNEGADGVGSSSGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 37 - protein, 7 aa, synthetic construct
VEAVGGN

SEQ ID NO: 38 - protein, 7 aa, synthetic construct
KSSKNAT

SEQ ID NO: 39 - protein, 7 aa, synthetic construct
CHERRRV

SEQ ID NO: 40 - protein, 7 aa, synthetic construct
PKSRAGA

SEQ ID NO: 41 - protein, 7 aa, synthetic construct
RARGDGG

SEQ ID NO: 42 - protein, 7 aa, synthetic construct
KVSNAAN

SEQ ID NO: 43 - protein, 7 aa, synthetic construct
VVKQRGD

SEQ ID NO: 44 - protein, 7 aa, synthetic construct
PADNVKA

SEQ ID NO: 45 - protein, 7 aa, synthetic construct
RDGGTKA

(19) United States
(12) Patent Application Publication
(10) Pub. No.: US 2026/0248958 A1
(43) Pub. Date: Aug. 27, 2026

(54) HUMAN CENTRAL NERVOUS SYSTEM (CNS) TARGETING AAV VARIANTS

(71) Applicants: CZ Biohub SF, LLC, San Francisco, CA (US); The Regents of the University of California, Oakland, CA (US)

(72) Inventors: David V. Schaffer, Danville, CA (US); DanQing Zhu, Oakland, CA (US); Tomasz J. Nowakowski, San Francisco, CA (US); Galina Popova, San Francisco, CA (US)

(21) Appl. No.: 19/159,544

(22) PCT Filed: Mar. 8, 2024
(86) PCT No.: PCT/US2024/019280

§ 371 Date: Aug. 25, 2025

Related U.S. Application Data
(60) Provisional application No. 63/452,118, filed on Mar. 14, 2023.

(51) Int. Cl. A61K 48/00; C07K 14/005; C12N 9/22; C12N 15/86

(52) U.S. Cl. CPC A61K 48/0033; C07K 14/005; C12N 9/226; C12N 15/86; C12N 2750/14122; C12N 2750/14143; C12N 2750/14145

(57) ABSTRACT

The present disclosure provides recombinant adeno-associated virus (AAV) virions with altered capsid protein, where the recombinant AAV (rAAV) virions exhibit greater infectivity of a central nervous system (CNS) cell, compared to wild-type rAAV, and where the rAAV virions comprise a heterologous nucleic acid. The present disclosure provides methods of delivering a gene product to a CNS cell in an individual.

### CROSS-REFERENCE

This application claims the benefit of U.S. Provisional Patent Application No. 63/452,118, filed Mar. 14, 2023, which application is incorporated herein by reference in its entirety.

### STATEMENT REGARDING FEDERALLY SPONSORED RESEARCH

This invention was made with government support under Grant Number MH130700 awarded by the National Institutes of Health. The government has certain rights in the invention.

### INCORPORATION-BY-REFERENCE OF MATERIAL ELECTRONICALLY SUBMITTED

A Sequence Listing is provided herewith as a Sequence Listing XML, “BERK-482WO_SEQ_LIST.XML” created on Mar. 5, 2024 and having a size of 345,013 bytes. The contents of the Sequence Listing XML are incorporated by reference herein in their entirety.

### INTRODUCTION

Adeno-associated virus (AAV) belongs to the Parvoviridae family and Dependovirus genus, whose members require co-infection with a helper virus such as adenovirus to promote replication, and AAV establishes a latent infection in the absence of a helper. AAV virions are composed of a 25 nm icosahedral capsid encompassing a 4.7 kb single-stranded DNA genome with two open reading frames: rep and cap. The non-structural rep gene encodes four regulatory proteins essential for viral replication, whereas cap encodes three structural proteins (VP1-3) that assemble into a 60-mer capsid shell. This viral capsid mediates the ability of AAV vectors to overcome many of the biological barriers of viral transduction-including cell surface receptor binding, endocytosis, intracellular trafficking, and unpackaging in the nucleus.

The central nervous system (CNS) comprises a multitude of cell types with diverse functionality and specialization. Dysregulation of neuronal or glial (including microglia) populations has been implicated in multiple disorders, including Alzheimer's, Parkinson's, Multiple Sclerosis, and Huntington's disease. AAVs hold tremendous promise as gene delivery vectors to treat such conditions given their reasonable starting efficiency and safety profile. However, such strategies remain challenging due to difficulties in efficient and targeted delivery to specific cell populations.

The present disclosure provides variant AAV that address these as well as other needs.

### SUMMARY

The present disclosure provides recombinant adeno-associated virus (rAAV) with an altered capsid protein, where the recombinant AAV (rAAV) exhibits greater ability to infect a CNS cell compared to wild-type AAV. rAAV that infect different types of CNS cells are provided. These rAAV disclosed herein include a variant capsid protein that mediates infection of different types of CNS cells. These rAAV can be used to deliver a nucleic acid to these cells.

### BRIEF DESCRIPTION OF THE DRAWINGS

FIG. 1. Directed evolution workflow for AAV vector engineering targeting specific CNS cell types.

FIG. 2. Transduction of human microglial cells in brain slices by natural AAV serotypes.

FIG. 3. Representative images of AAV-GFP transduction towards neuronal cells using evolved AAV variants. A. Examples of variant 1 (KSSKNAT; SEQ ID NO:38), variant 2 (CHERRRV; SEQ ID NO:39), variant 6 (PKSRAGA; SEQ ID NO:40), and variant 8 (RARGDGG; SEQ ID NO:41) infecting neuronal cells in prenatal brain slices. Neurons are enriched in the upper layers of developing cortex, CP and SP. CP—cortical plate, SP—subplate, IZ—intermediate zone, GZ—germinal zone. GFP-positive cells (in green) are found predominantly in the areas with high expression of a neuron-specific marker Huc/HuD (in red). B. Zoomed-in regions from A, variant 1, from cortical plate (top) or subplate (bottom) showing co-localization with neuronal marker HuC and typical neuronal morphology of the cells.

FIG. 4. Representative images of AAV-GFP transduction towards glial cells using evolved AAV variants. A. Glial variant 3 (KVSNAAN; SEQ ID NO:42) and glial variant 4 (G4: VVKQRGD; SEQ ID NO:43) in prenatal human tissue, ventricular zone. dsRed-expressing AAV was used to label glial cells. dsRed-positive cells (in red) are predominantly localized in the germinal zone rich in glial cells and co-localize with GFAP-positive glial cells (green). B. Variant 4 in adult human brain tissue in white matter. Oligodendrocytes are labeled with Olig2 (in green).

FIG. 5. Immunostaining showed high levels of glial infection using evolved AAV variants across multiple regions of the primary brain tissue. CP: Cortical Plate; IZ: Intermediate Zone; VZ: Ventricular Zone.

FIG. 6. Representative images of AAV-GFP transduction towards microglia cells using evolved AAV variants. Adult human brain tissue expressing GFP (green) in microglia cells labeled with Iba1 (red). Var 1: PADNVKA (SEQ ID NO:44); Var 2: RDGGTKA (SEQ ID NO: 45).

FIG. 7. Representative images of AAV-GFP transduction various glial cell types from ML7 variant (7-mer peptide, KVTRGDT (SEQ ID NO:10), inserted in capsid protein) under CMV promoter. Prenatal human brain tissue expressing GFP (green) in cells co-expressing glial marker Sox9 in the cortical plate, where astrocytes reside (left), and in ventricular zone rich in radial glial cells (right). Cortical plate cells display typical astrocytic morphology.

FIG. 8. Depicts amino acid sequences of GH loop region of the VP1 protein of the indicated AAVs.

FIG. 9. The N5 variant (GFTDDAT (SEQ ID NO:20)) infects neurons (in human cortical slices) selectively and very efficiently at a MOI of 1000 (top row), and the G4 variant (VVKQRGD (SEQ ID NO:43)) infects astrocytes selectively and efficiently (bottom row).

### DEFINITIONS

“AAV” is an abbreviation for adeno-associated virus, and may be used to refer to the virus itself or derivatives thereof. The term covers all subtypes and both naturally occurring and variants, except where required otherwise. The abbreviation “rAAV” refers to recombinant adeno-associated virus (also referred to as virions or viral particles) that may include a heterologous nucleic acid for delivering into a cell for, e.g., gene therapy. The term “AAV” includes naturally occurring serotypes and derivatives thereof. The term “AAV” includes AAV type 1 (AAV-1), AAV type 2 (AAV-2), AAV type 3 (AAV-3), AAV type 4 (AAV-4), AAV type 5 (AAV-5), AAV type 6 (AAV-6), AAV type 7 (AAV-7), AAV type 8 (AAV-8), AAV type 9 (AAV-9), AAV type 10 (AAV-10), AAV type 11 (AAV-11), avian AAV, bovine AAV, canine AAV, equine AAV, primate AAV, non-primate AAV, ovine AAV, and the like. See, e.g., Mori et al. (2004) Virology 330:375. The term “AAV” also includes chimeric AAV. “Primate AAV” refers to AAV isolated from a primate, “non-primate AAV” refers to AAV isolated from a non-primate mammal, “bovine AAV” refers to AAV isolated from a bovine mammal (e.g., a cow), etc.

An “rAAV vector” as used herein refers to a recombinant AAV vector comprising a polynucleotide sequence not of AAV origin (i.e., a polynucleotide heterologous to AAV), typically a sequence of interest for the genetic transformation of a cell. In general, the heterologous polynucleotide is flanked by at least one, and generally by two AAV inverted terminal repeat sequences (ITRs). The term rAAV vector encompasses both rAAV vector particles and rAAV vector plasmids. The AAV ITR sequences may be obtained from any known AAV, including presently identified mammalian AAV types. As non-limiting examples, the 5′ and 3′ AAV ITR sequences may be AAV1-AAV9 5′ and 3′ ITR sequences, respectively, e.g., AAV5 5′ and 3′ ITR sequences, respectively, or AAV2 5′ and 3′ ITR sequences, respectively.

An “rAAV” or “rAAV particle” or “rAAV vector particle” or “rAAV virion” refers to a viral particle composed of at least one AAV capsid protein (typically by all of the capsid proteins which may all be wild type or may include variants of wild type capsid protein(s)) and an encapsidated polynucleotide rAAV vector. If the particle comprises a heterologous polynucleotide (i.e., a polynucleotide other than a wild-type AAV genome, such as a transgene to be delivered to a mammalian cell), it is typically referred to as an “rAAV vector particle” or simply an “rAAV vector”. Thus, production of rAAV (also referred to as a rAAV particle or virion) necessarily includes production of rAAV vector, as such a vector is contained within an rAAV particle.

“Packaging” refers to a series of intracellular events that result in the assembly of capsid proteins and encapsidation of a rAAV vector and generation of a rAAV particle.

AAV “rep” and “cap” genes refer to polynucleotide sequences encoding replication and encapsidation (also referred to as capsid) proteins of adeno-associated virus. AAV rep and cap are referred to herein as AAV “packaging genes.”

A “helper virus” for AAV refers to a virus or parts thereof (e.g., helper proteins and/or VA RNA) that allow AAV to be replicated and packaged by a mammalian cell. A variety of such helper viruses for AAV are known in the art, including adenoviruses, herpesviruses and poxviruses such as vaccinia. The adenoviruses encompass a number of different subgroups, although Adenovirus type 5 of subgroup C is most commonly used. Numerous adenoviruses of human, non-human mammalian and avian origin are known and available from depositories such as the ATCC. Viruses of the herpes family include, for example, herpes simplex viruses (HSV) and Epstein-Barr viruses (EBV), as well as cytomegaloviruses (CMV) and pseudorabies viruses (PRV); which are also available from depositories such as ATCC.

“Helper virus function(s)” refers to function(s) encoded in a helper virus genome which allow AAV replication and packaging (in conjunction with other requirements for replication and packaging described herein). As described herein, “helper virus function” may be provided in a number of ways, including by providing helper virus or providing, for example, polynucleotide sequences encoding the requisite function(s) to a producer cell in trans.

An “infectious” virus or viral particle is one that is capable of attaching to a cell and inserting into the cell the polynucleotide encapsidated in the virus. The term does not necessarily imply any replication capacity of the virus. As used herein, an “infectious” virus or viral particle is one that can access a target cell, can infect a target cell, and can express a heterologous nucleic acid in a target cell. Thus, “infectivity” refers to the ability of a virus to access a target cell, infect a target cell, and deliver a nucleic acid in a target cell. Infectivity can refer to in vitro infectivity or in vivo infectivity. Assays for counting infectious viral particles are described elsewhere in this disclosure and in the art. Viral infectivity can be expressed as the ratio of infectious viral particles to total viral particles. Total viral particles can be expressed as the number of viral genome (vg) copies. The ability of a virus to express a heterologous nucleic acid in a cell can be referred to as “transduction.” The ability of a viral particle to express a heterologous nucleic acid in a cell can be assayed using a number of techniques, including assessment of a marker gene, such as a green fluorescent protein (GFP) assay (e.g., where the virus comprises a nucleotide sequence encoding GFP), where GFP is produced in a cell infected with the viral particle and is detected and/or measured; or the measurement of a produced protein, for example by an enzyme-linked immunosorbent assay (ELISA). Viral infectivity can be expressed as the ratio of infectious viral particles to total viral particles. Methods of determining the ratio of infectious viral particle to total viral particle are known in the art. See, e.g., Grainger et al. (2005) Mol. Ther. 11: S337 (describing a TCID50 infectious titer assay); and Zolotukhin et al. (1999) Gene Ther. 6:973.

A “replication-competent” virus (e.g., a replication-competent AAV) refers to a phenotypically wild-type virus that is infectious, and is also capable of being replicated in an infected cell (i.e., in the presence of a helper virus or helper virus functions). In the case of AAV, replication competence generally requires the presence of functional AAV packaging genes. In general, rAAV vectors as described herein are replication-incompetent in mammalian cells (especially in human cells) by virtue of the lack of one or more AAV packaging genes. Typically, such rAAV vectors lack any AAV packaging gene sequences in order to minimize the possibility that replication competent AAV are generated by recombination between AAV packaging genes and an incoming rAAV vector. In many embodiments, the AAV provided herein are those which contain few if any replication competent AAV (rcAAV, also referred to as RCA) (e.g., less than about 1 rcAAV per 10² rAAV particles, less than about 1 rcAAV per 10⁴ rAAV particles, less than about 1 rcAAV per 10⁸ rAAV particles, less than about 1 rcAAV per 10¹² rAAV particles, or no rcAAV).

The term “polynucleotide” refers to a polymeric form of nucleotides of any length, including deoxyribonucleotides or ribonucleotides, or analogs thereof. A polynucleotide may comprise modified nucleotides, such as methylated nucleotides and nucleotide analogs, and may be interrupted by non-nucleotide components. If present, modifications to the nucleotide structure may be imparted before or after assembly of the polymer. The term polynucleotide, as used herein, refers interchangeably to double- and single-stranded molecules. Unless otherwise specified or required, any embodiment of the invention described herein that is a polynucleotide encompasses both the double-stranded form and each of two complementary single-stranded forms known or predicted to make up the double-stranded form.

A polynucleotide or polypeptide has a certain percent “sequence identity” to another polynucleotide or polypeptide, meaning that, when aligned, that percentage of bases or amino acids are the same when comparing the two sequences. Sequence similarity can be determined in a number of different manners. To determine sequence identity, sequences can be aligned using the methods and computer programs, including BLAST, available over the world wide web at ncbi.nlm.nih.gov/BLAST/. Another alignment algorithm is FASTA, available in the Genetics Computing Group (GCG) package, from Madison, Wisconsin, USA, a wholly owned subsidiary of Oxford Molecular Group, Inc. Other techniques for alignment are described in Methods in Enzymology, vol. 266: Computer Methods for Macromolecular Sequence Analysis (1996), cd. Doolittle, Academic Press, Inc., a division of Harcourt Brace & Co., San Diego, California, USA. Of particular interest are alignment programs that permit gaps in the sequence. The Smith-Waterman is one type of algorithm that permits gaps in sequence alignments. See Meth. Mol. Biol. 70:173-187 (1997). Also, the GAP program using the Needleman and Wunsch alignment method can be utilized to align sequences. See J. Mol. Biol. 48:443-453 (1970)

Of interest is the BestFit program using the local homology algorithm of Smith Waterman (Advances in Applied Mathematics 2:482-489 (1981) to determine sequence identity. The gap generation penalty will generally range from 1 to 5, usually 2 to 4 and in many embodiments will be 3. The gap extension penalty will generally range from about 0.01 to 0.20 and in many instances will be 0.10. The program has default parameters determined by the sequences inputted to be compared. Preferably, the sequence identity is determined using the default parameters determined by the program. This program is available also from Genetics Computing Group (GCG) package, from Madison, Wisconsin, USA.

Another program of interest is the FastDB algorithm. FastDB is described in Current Methods in Sequence Comparison and Analysis, Macromolecule Sequencing and Synthesis, Selected Methods and Applications, pp. 127-149, 1988, Alan R. Liss, Inc. Percent sequence identity is calculated by FastDB based upon the following parameters:
Mismatch Penalty: 1.00;
Gap Penalty: 1.00;
Gap Size Penalty: 0.33; and
Joining Penalty: 30.0.

A “gene” refers to a polynucleotide containing at least one open reading frame that is capable of encoding a particular protein after being transcribed and translated.

The term “guide RNA”, as used herein, refers to an RNA that comprises: i) an “activator” nucleotide sequence that binds to a guide RNA-directed endonuclease (e.g., a class 2 CRISPR/Cas endonuclease such as a type II, type V, or type VI CRISPR/Cas endonuclease) and activates the RNA-directed endonuclease; and ii) a “targeter” nucleotide sequence that comprises a nucleotide sequence that hybridizes with a target nucleic acid. The “activator” nucleotide sequence and the “targeter” nucleotide sequence can be on separate RNA molecules (e.g., a “dual-guide RNA”); or can be on the same RNA molecule (a “single-guide RNA”).

A “small interfering” or “short interfering RNA” or siRNA is an RNA duplex of nucleotides that is targeted to a gene interest (a “target gene”). An “RNA duplex” refers to the structure formed by the complementary pairing between two regions of an RNA molecule. siRNA is “targeted” to a gene in that the nucleotide sequence of the duplex portion of the siRNA is complementary to a nucleotide sequence of the targeted gene. In some embodiments, the length of the duplex of siRNAs is less than 30 nucleotides. In some embodiments, the duplex can be 29, 28, 27, 26, 25, 24, 23, 22, 21, 20, 19, 18, 17, 16, 15, 14, 13, 12, 11 or 10 nucleotides in length. In some embodiments, the length of the duplex is 19-25 nucleotides in length. The RNA duplex portion of the siRNA can be part of a hairpin structure. In addition to the duplex portion, the hairpin structure may contain a loop portion positioned between the two sequences that form the duplex. The loop can vary in length. In some embodiments the loop is 5, 6, 7, 8, 9, 10, 11, 12 or 13 nucleotides in length. The hairpin structure can also contain 3′ or 5′ overhang portions. In some embodiments, the overhang is a 3′ or a 5′ overhang 0, 1, 2, 3, 4 or 5 nucleotides in length.

As used herein, the term “microRNA” refers to any type of interfering RNAs, including but not limited to, endogenous microRNAs and artificial microRNAs (e.g., synthetic miRNAs). Endogenous microRNAs are small RNAs naturally encoded in the genome which are capable of modulating the productive utilization of mRNA. An artificial microRNA can be any type of RNA sequence, other than endogenous microRNA, which is capable of modulating the activity of an mRNA. A microRNA sequence can be an RNA molecule composed of any one or more of these sequences. MicroRNA (or “miRNA”) sequences have been described in publications such as Lim, et al., 2003, Genes & Development, 17, 991-1008, Lim et al., 2003, Science, 299, 1540, Lee and Ambrose, 2001, Science, 294, 862, Lau et al., 2001, Science 294, 858-861, Lagos-Quintana et al., 2002, Current Biology, 12, 735-739, Lagos-Quintana et al., 2001, Science, 294, 853-857, and Lagos-Quintana et al., 2003, RNA, 9, 175-179. Examples of microRNAs include any RNA that is a fragment of a larger RNA or is a miRNA, siRNA, stRNA, sncRNA, tncRNA, snoRNA, smRNA, shRNA, snRNA, or other small non-coding RNA. See, e.g., US Patent Applications 20050272923, 20050266552, 20050142581, and 20050075492. A “microRNA precursor” (or “pre-miRNA”) refers to a nucleic acid having a stem-loop structure with a microRNA sequence incorporated therein. A “mature microRNA” (or “mature miRNA”) includes a microRNA that has been cleaved from a microRNA precursor (a “pre-miRNA”), or that has been synthesized (e.g., synthesized in a laboratory by cell-free synthesis), and has a length of from about 19 nucleotides to about 27 nucleotides, e.g., a mature microRNA can have a length of 19 nt, 20 nt, 21 nt, 22 nt, 23 nt, 24 nt, 25 nt, 26 nt, or 27 nt. A mature microRNA can bind to a target mRNA and inhibit translation of the target mRNA.

“Recombinant,” as applied to a polynucleotide means that the polynucleotide is the product of various combinations of cloning, restriction or ligation steps, and other procedures that result in a construct that is distinct from a polynucleotide found in nature. A recombinant virus is a viral particle comprising a recombinant polynucleotide. The terms respectively include replicates of the original polynucleotide construct and progeny of the original virus construct.

A “control element” or “control sequence” is a nucleotide sequence involved in an interaction of molecules that contributes to the functional regulation of a polynucleotide, including replication, duplication, transcription, splicing, translation, or degradation of the polynucleotide. The regulation may affect the frequency, speed, or specificity of the process, and may be enhancing or inhibitory in nature. Control elements known in the art include, for example, transcriptional regulatory sequences such as promoters and enhancers. A promoter is a DNA region capable under certain conditions of binding RNA polymerase and initiating transcription of a coding region usually located downstream (in the 3′ direction) from the promoter.

“Operatively linked” or “operably linked” refers to a juxtaposition of genetic elements, wherein the elements are in a relationship permitting them to operate in the expected manner. For instance, a promoter is operatively linked to a coding region if the promoter helps initiate transcription of the coding sequence. There may be intervening residues between the promoter and coding region so long as this functional relationship is maintained.

An “expression vector” is a vector comprising a region which encodes a polypeptide of interest, and is used for effecting the expression of the protein in an intended target cell. An expression vector also comprises control elements operatively linked to the encoding region to facilitate expression of the protein in the target. The combination of control elements and a gene or genes to which they are operably linked for expression is sometimes referred to as an “expression cassette,” a large number of which are known and available in the art or can be readily constructed from components that are available in the art.

“Heterologous” means derived from a genotypically distinct entity from that of the rest of the entity to which it is being compared. For example, a polynucleotide introduced by genetic engineering techniques into a plasmid or vector derived from a different species is a heterologous polynucleotide. A promoter removed from its native coding sequence and operatively linked to a coding sequence with which it is not naturally found linked is a heterologous promoter. Thus, for example, an rAAV that includes a heterologous nucleic acid encoding a heterologous gene product is an rAAV that includes a nucleic acid not normally included in a naturally-occurring, wild-type AAV, and the encoded heterologous gene product is a gene product not normally encoded by a naturally-occurring, wild-type AAV. As another example, a variant AAV capsid protein that comprises a heterologous peptide inserted into the GH loop of the capsid protein is a variant AAV capsid protein that includes an insertion of a peptide not normally included in a naturally-occurring, wild-type AAV.

The terms “genetic alteration” and “genetic modification” (and grammatical variants thereof), are used interchangeably herein to refer to a process wherein a genetic element (e.g., a polynucleotide) is introduced into a cell other than by mitosis or meiosis. The element may be heterologous to the cell, or it may be an additional copy or improved version of an element already present in the cell. Genetic alteration may be effected, for example, by transfecting a cell with a recombinant plasmid or other polynucleotide through any process known in the art, such as electroporation, calcium phosphate precipitation, or contacting with a polynucleotide-liposome complex. Genetic alteration may also be effected, for example, by transduction or infection with a DNA or RNA virus or viral vector. Generally, the genetic element is introduced into a chromosome or mini-chromosome in the cell; but any alteration that changes the phenotype and/or genotype of the cell and its progeny is included in this term.

A cell is said to be “stably” altered, transduced, genetically modified, or transformed with a genetic sequence if the sequence is available to perform its function during extended culture of the cell in vitro. Generally, such a cell is “heritably” altered (genetically modified) in that a genetic alteration is introduced which is also inheritable by progeny of the altered cell.

The terms “polypeptide,” “peptide,” and “protein” are used interchangeably herein to refer to polymers of amino acids of any length. The terms also encompass an amino acid polymer that has been modified; for example, disulfide bond formation, glycosylation, lipidation, phosphorylation, or conjugation with a labeling component. Polypeptides such as anti-angiogenic polypeptides, neuroprotective polypeptides, and the like, when discussed in the context of delivering a gene product to a mammalian subject, and compositions therefor, refer to the respective intact polypeptide, or any fragment or genetically engineered derivative thereof, which retains the desired biochemical function of the intact protein. Similarly, references to nucleic acids encoding anti-angiogenic polypeptides, nucleic acids encoding neuroprotective polypeptides, and other such nucleic acids for use in delivery of a gene product to a mammalian subject (which may be referred to as “transgenes” to be delivered to a recipient cell), include polynucleotides encoding the intact polypeptide or any fragment or genetically engineered derivative possessing the desired biochemical function.

An “isolated” plasmid, nucleic acid, vector, virus, virion, host cell, or other substance refers to a preparation of the substance devoid of at least some of the other components that may also be present where the substance or a similar substance naturally occurs or is initially prepared from. Thus, for example, an isolated substance may be prepared by using a purification technique to enrich it from a source mixture. Enrichment can be measured on an absolute basis, such as weight per volume of solution, or it can be measured in relation to a second, potentially interfering substance present in the source mixture. Increasing enrichments of the embodiments of this invention are increasingly more isolated. An isolated plasmid, nucleic acid, vector, virus, host cell, or other substance is in some embodiments purified, e.g., from about 80% to about 90% pure, at least about 90% pure, at least about 95% pure, at least about 98% pure, or at least about 99%, or more, pure.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment,” as used herein, covers any treatment of a disease in a mammal, particularly in a human, and includes: (a) preventing the disease from occurring in a subject which may be predisposed to the disease or at risk of acquiring the disease but has not yet been diagnosed as having it; (b) inhibiting the disease, i.e., arresting its development; and (c) relieving the disease, i.e., causing regression of the disease.

The terms “individual,” “host,” “subject,” and “patient” are used interchangeably herein, and refer to a mammal, including, but not limited to, human and non-human primates, including simians and humans; mammalian sport animals (e.g., horses, camels, etc.); mammalian farm animals (e.g., sheep, goats, cows, etc.); mammalian pets (dogs, cats, etc.); and rodents (e.g., mice, rats, etc.). In some cases, the individual is a human.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “an rAAV virion” includes a plurality of such virions and reference to “the variant capsid protein” includes reference to one or more variant capsid proteins and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides recombinant adeno-associated virus (rAAV) with an altered capsid protein, where the recombinant AAV (rAAV) exhibits greater ability to infect a CNS cell compared to wild-type AAV. rAAV that infect different types of CNS cells are provided. These rAAV disclosed herein include a variant capsid protein that mediates infection of different types of CNS cells. These rAAV can be used to deliver a nucleic acid to these cells The present disclosure provides methods of delivering a gene product to a CNS cell in a subject. The present disclosure also provides methods of modifying a target nucleic acid present in a CNS cell in a subject.

The present disclosure provides recombinant adeno-associated virus (AAV) with altered capsid protein, where the recombinant AAV (rAAV) exhibit greater infectivity of a CNS cell compared to a control rAAV with the unaltered capsid protein; and where the rAAV comprise a heterologous nucleic acid. The CNS cell can be a glia, neuron, or microglia. The present disclosure further provides methods of delivering a gene product to a CNS cell in a subject, and methods of treating a CNS disease. The present disclosure provides an rAAV with an altered capsid protein, where the rAAV exhibits at least 5-fold increased localization to one or more of a glial cell, a neuron, or a microglial cell, compared to the extent of localization to a glial cell, a neuron, or a microglial cell, by a control rAAV comprising the corresponding parental AAV capsid protein; and where the control rAAV comprise the heterologous nucleic acid.

### Variant AAV Capsid Polypeptides

The present disclosure provides a variant AAV capsid protein. As noted above, a variant AAV capsid protein of the present disclosure is altered, compared to a parental capsid protein which may be wild-type or other reference AAV capsid protein. Alterations include insertions of a contiguous stretch of amino acids.

In some cases, a variant AAV capsid protein of the present disclosure comprises an insertion of a heterologous peptide of from 7 amino acids to 20 amino acids in length in an insertion site in a surface-accessible (e.g., solvent-accessible) portion of a parental AAV capsid protein, such that the variant capsid protein, when present in an AAV, confers increased infectivity of a CNS cell compared to the infectivity of the CNS cell by a control rAAV virion comprising the corresponding parental AAV capsid protein. An “insertion of from about 7 amino acids to about 20 amino acids” is also referred to herein as a “peptide insertion” (e.g., a heterologous peptide insertion). A “corresponding parental AAV capsid protein” refers to an AAV capsid protein before insertion of a heterologous peptide insertion. In some instances, the variant AAV capsid comprises a single heterologous peptide insert of from 7 amino acids to 20 amino acids (e.g., from 7 to 10, from 10 to 12, from 12 to 15, or from 15 to 20 amino acids) in length.

The insertion site may be in the GH loop, or loop IV, of the AAV capsid protein, e.g., in a solvent-accessible portion of the GH loop, or loop IV, of the AAV capsid protein. For the GH loop/loop IV of AAV capsid, see, e.g., van Vliet et al. (2006) Mol. Ther. 14:809; Padron et al. (2005) J. Virol. 79:5047; and Shen et al. (2007) Mol. Ther. 15:1955. For example, the insertion site can be within amino acids 560-601 of an AAV5 capsid protein or the corresponding amino acids in another AAV capsid protein, e.g., AAV1, AAV2, AAV2G9, AAV3, AAV3a, AAV3b, AAV3-3, AAV4, AAV4-4, AAV6, AAV6.1, AAV6.2, AAV6.1.2, AAV7, AAV7.2, AAV8, AAV9, AAV9.11, AAV9.13, AAV9.16, AAV9.24, AAV9.45, AAV9.47, AAV9.61, AAV9.68, AAV9.84, AAV9.9, AAV10, AAV11, or AAV12. Amino acids 560-601 of an AAV5 capsid protein has the following sequence: RVAYNVGGQMATNNQSSTTAPATGTYNLQEIVPGSVWMERDV (SEQ ID NO:11). For example, the insertion site can be within amino acids 575-577 of AAV5 or a corresponding insertion site in an AAV of a different serotype. In some cases, the insertion site is between amino acids 575 and 576 of an AAV5 capsid protein, or a corresponding insertion site in an AAV of a different serotype. In some cases, the insertion site is between amino acids 576 and 577 of an AAV5 capsid protein, or a corresponding insertion site in an AAV of a different serotype. In some cases, the insertion site is between amino acids 575 and 577 of an AAV5 capsid protein, or a corresponding insertion site in an AAV of a different serotype, where the amino acid at position 576 is replaced with the heterologous peptide.

In some cases, the insertion site is between amino acids 587 and 588 of an AAV2 capsid protein. In some cases, the insertion site is between amino acids 584 and 585 of an AAV4 capsid protein.

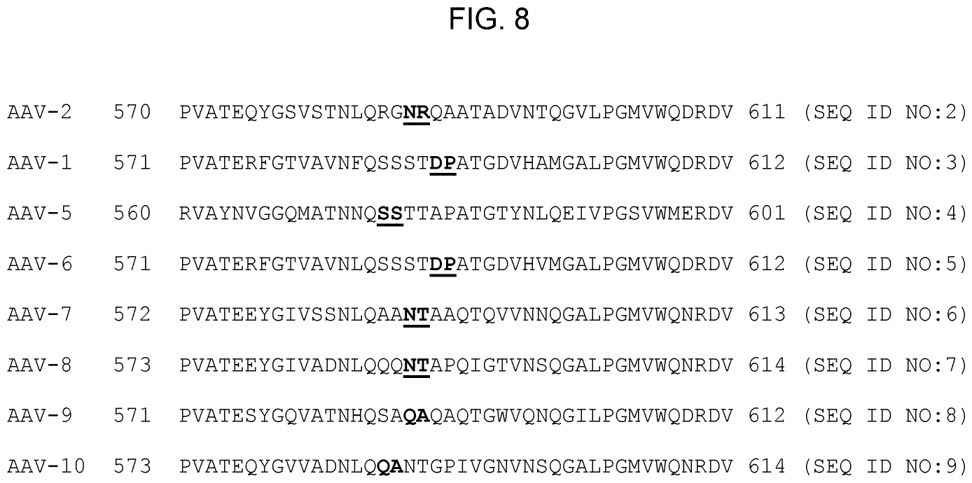

Sequences corresponding to amino acids 560-601 of capsid protein VP1 of AAV5 in various AAV serotypes are shown in FIG. 8. Exemplary insertion sites are underlined in FIG. 8; the amino acid numbering is based on the numbering depicted in FIG. 8. Full length VP1 sequences are available at, e.g., GenBank Accession No. NP_049542 for AAV1; GenBank Accession No. NP_044927 for AAV4; GenBank Accession No. AAD13756 for AAV5; GenBank Accession No. AAB95459 for AAV6; GenBank Accession No. YP_077178 for AAV7; GenBank Accession No. YP_077180 for AAV8; GenBank Accession No. AAS99264 for AAV9; GenBank Accession No. AAT46337 for AAV10; and GenBank Accession No. AAO88208 for AAVrh10. See, e.g., Santiago-Ortiz et al. (2015) Gene Ther. 22:934 for ancestral AAV capsid.

### Insertion Peptides

As noted above, a heterologous peptide of from about 7 amino acids to about 20 amino acids in length is inserted into the GH loop of an AAV capsid. In some cases, the insertion peptide has a length of from 7 amino acids to 20 amino acids. In some cases, the insertion peptide has a length of from 7 amino acids to 15 amino acids. In some cases, the insertion peptide has a length of from 9 amino acids to 15 amino acids. In some cases, the insertion peptide has a length of from 9 amino acids to 12 amino acids. The insertion peptide has a length of 7 amino acids, 8 amino acids, 9 amino acids, 10 amino acids, 11 amino acids, 12 amino acids, 13 amino acids, 14 amino acids, 15 amino acids, 16 amino acids, 17 amino acids, 18 amino acids, 19 amino acids, or 20 amino acids. In some cases, the insertion peptide has a length of 7 amino acids. In some cases, the insertion peptide has a length of 8 amino acids. In some cases, the insertion peptide has a length of 9 amino acids. In some cases, the insertion peptide has a length of 10 amino acids. In some cases, the insertion peptide has a length of 11 amino acids. In some cases, the insertion peptide has a length of 12 amino acids. In some cases, the insertion peptide has a length of 13 amino acids. In some cases, the insertion peptide has a length of 14 amino acids. In some cases, the insertion peptide has a length of 15 amino acids.

The peptide insert, in some cases, includes the sequence:

```
  1)
(SEQ ID NO: 42)
KVSNAAN;
  2)
(SEQ ID NO: 43)
VVKQRGD;
  3)
(SEQ ID NO: 12)
VTNVVRA;
  4)
(SEQ ID NO: 13)
PGPGNTI;
  5)
(SEQ ID NO: 14)
QRIVNEV;
  6)
(SEQ ID NO: 38)
KSSKNAT;
  7)
(SEQ ID NO: 40)
PKSRAGA;
  8)
(SEQ ID NO: 41)
RARGDGG;
  9)
(SEQ ID NO: 16)
RSSNYVV;
10)
(SEQ ID NO: 17)
AAPIDGE;
11)
(SEQ ID NO: 18)
IGPVAAD;
12)
(SEQ ID NO: 19)
DGVEDAV;
13)
(SEQ ID NO: 20)
GFTDDAT;
14)
(SEQ ID NO: 21)
LEAVATV;
15)
(SEQ ID NO: 10)
KVTRGDT;
16)
(SEQ ID NO: 44)
PADNVKA;
17)
(SEQ ID NO: 45)
RDGGTKA;
18)
(SEQ ID NO: 22)
VEAVGGNVEAVGGN;
19)
(SEQ ID NO: 23)
MSLLPYP;
20)
(SEQ ID NO: 24)
YFAIYIF;
21)
(SEQ ID NO: 25)
HLSDARP;
22)
(SEQ ID NO: 39)
CHERRRV;
23)
(SEQ ID NO: 26)
LIASFVQ;
24)
(SEQ ID NO: 27)
AGGGGKA;
25)
(SEQ ID NO: 28)
KGVQERA;
or
26)
(SEQ ID NO: 29)
AVRINPG.
```

In some cases, the capsid protein when present in a rAAV confers to the rAAV increased infectivity of a glial cell compared to the infectivity of the glial cell by a control rAAV comprising the corresponding parental AAV capsid protein and may include a heterologous peptide comprising the amino acid sequence: 1) KVSNAAN (SEQ ID NO:42); 2) VVKQRGD (SEQ ID NO:43); 3) VTNVVRA (SEQ ID NO:12); 4) PGPGNTI (SEQ ID NO:13); or 5) QRIVNEV (SEQ ID NO:14).

In some cases, the capsid protein when present in a rAAV confers to the rAAV increased infectivity of a neuron compared to the infectivity of the neuron by a control rAAV comprising the corresponding parental AAV capsid protein and may include a heterologous peptide comprising the amino acid sequence: 6) KSSKNAT (SEQ ID NO:38); 7) PKSRAGA (SEQ ID NO: 40); 8) RARGDGG (SEQ ID NO:41); 9) RSSNYVV (SEQ ID NO:16); 10) AAPIDGE (SEQ ID NO: 17); 11) IGPVAAD (SEQ ID NO:18); 12) DGVEDAV (SEQ ID NO:19); 13) GFTDDAT (SEQ ID NO:20); or 14) LEAVATV (SEQ ID NO:21).

In some cases, the capsid protein when present in a rAAV confers to the rAAV increased infectivity of a microglia compared to the infectivity of the microglia by a control rAAV comprising the corresponding parental AAV capsid protein and may include a heterologous peptide comprising the amino acid sequence: 15) KVTRGDT (SEQ ID NO:10); 16) PADNVKA (SEQ ID NO:44); 17) RDGGTKA (SEQ ID NO:45); 18) VEAVGGNVEAVGGN (SEQ ID NO: 22); 19) MSLLPYP (SEQ ID NO:23); 20) YFAIYIF (SEQ ID NO:24); 21) HLSDARP (SEQ ID NO: 25); 22) CHERRRV (SEQ ID NO:39); 23) LIASFVQ (SEQ ID NO:26); 24) AGGGGKA (SEQ ID NO:27); 25) KGVQERA (SEQ ID NO:28); or 26) AVRINPG (SEQ ID NO:29).

In some cases, a variant AAV capsid polypeptide of the present disclosure is a chimeric capsid, e.g., the capsid comprises a portion of an AAV capsid of a first AAV serotype and a portion of an AAV capsid of a second serotype; and comprises an insertion of from about 7 amino acids to about 20 amino acids (e.g., 5, 6, 7, 8, 9, 10, 11, 12, 13, 14, 15, 16, 17, 18, 19, or 20 amino acids; e.g., 9 amino acids, 10 amino acids, 11 amino acids, or 12 amino acids) in the GH loop or loop IV relative to a corresponding parental AAV capsid protein.

In some cases, a variant AAV capsid polypeptide of the present disclosure comprises an insertion of a heterologous peptides as provided herein in AAV5 capsid polypeptide, where the AAV5 capsid polypeptide is at least 85%, 90%, 95%, 97%, 98%, 99% or 100% identical to the wild type AAV5 capsid polypeptide. The wild type AAV5 capsid polypeptide may have the following amino acid sequence:

(SEQ ID NO: 1)
MSFVDHPPDWLEEVGEGLREFLGLEAGPPKPKPNQQHQDQARGLVLPGYNYLG
PGNGLDRGEPVNRADEVAREHDISYNEQLEAGDNPYLKYNHADAEFQEKLADDTSFGG
NLGKAVFQAKKRVLEPFGLVEEGAKTAPTGKRIDDHFPKRKKARTEEDSKPSTSSDAEA
GPSGSQQLQIPAQPASSLGADTMSAGGGGPLGDNNQGADGVGNASGDWHCDSTWMG
DRVVTKSTRTWVLPSYNNHQYREIKSGSVDGSNANAYFGYSTPWGYFDFNRFHSHWSP
RDWQRLINNYWGFRPRSLRVKIFNIQVKEVTVQDSTTTIANNLTSTVQVFTDDDYQLPY
VVGNGTEGCLPAFPPQVFTLPQYGYATLNRDNTENPTERSSFFCLEYFPSKMLRTGNNFE
FTYNFEEVPFHSSFAPSQNLFKLANPLVDQYLYRFVSTNNTGGVQFNKNLAGRYANTYK
NWFPGPMGRTQGWNLGSGVNRASVSAFATTNRMELEGASYQVPPQPNGMTNNLQGSN
TYALENTMIFNSQPANPGTTATYLEGNMLITSESETQPVNRVAYNVGGQMATNNQSSTT
APATGTYNLQEIVPGSVWMERDVYLQGPIWAKIPETGAHFHPSPAMGGFGLKHPPPMM
LIKNTPVPGNITSFSDVPVSSFITQYSTGQVTVEMEWELKKENSKRWNPEIQYTNNYNDP
QFVDFAPDSTGEYRTTRPIGTRYLTRPL.

In some cases, a variant AAV capsid polypeptide of the present disclosure comprises an insertion of a heterologous peptides as provided herein in AAV9 capsid polypeptide, where the AAV9 capsid polypeptide is at least 85%, 90%, 95%, 97%, 98%, 99% or 100% identical to the wild type AAV9 capsid polypeptide. The wild type AAV9 capsid polypeptide may have the following amino acid sequence:

(SEQ ID NO: 30)
MAADGYLPDWLEDNLSEGIREWWALKPGAPQPKANQQHQDNARGLVLPGYK
YLGPGNGLDKGEPVNAADAAALEHDKAYDQQLKAGDNPYLKYNHADAEFQERLKED
TSFGGNLGRAVFQAKKRLLEPLGLVEEAAKTAPGKKRPVEQSPQEPDSSAGIGKSGAQP
AKKRLNFGQTGDTESVPDPQPIGEPPAAPSGVGSLTMASGGGAPVADNNEGADGVGSSS
GNWHCDSQWLGDRVITTSTRTWALPTYNNHLYKQISNSTSGGSSNDNAYFGYSTPWGY
FDFNRFHCHFSPRDWQRLINNNWGFRPKRLNFKLFNIQVKEVTDNNGVKTIANNLTSTV
QVFTDSDYQLPYVLGSAHEGCLPPFPADVFMIPQYGYLTLNDGSQAVGRSSFYCLEYFPS
QMLRTGNNFQFSYEFENVPFHSSYAHSQSLDRLMNPLIDQYLYYLSKTINGSGQNQQTL
KFSVAGPSNMAVQGRNYIPGPSYRQQRVSTTVTQNNNSEFAWPGASSWALNGRNSLMN
PGPAMASHKEGEDRFFPLSGSLIFGKQGTGRDNVDADKVMITNEEEIKTTNPVATESYG
QVATNHQSAQAQAQTGWVQNQGILPGMVWQDRDVYLQGPIWAKIPHTDGNFHPSPLM
GGFGMKHPPPQILIKNTPVPADPPTAFNKDKLNSFITQYSTGQVSVEIEWELQKENSKRW
NPEIQYTSNYYKSNNVEFAVNTEGVYSEPRPIGTRYLTRNL.

In some cases, a variant AAV capsid polypeptide of the present disclosure comprises an insertion of a heterologous peptides as provided herein in an AAV capsid protein derived from AAV5 capsid protein where the AAV capsid protein retains the ability to confer to an rAAV comprising the AAV protein infectivity of a CNS cell, e.g., a glia, a neuron, or a microglia.

### Recombinant AAV Virions

The present disclosure provides a recombinant AAV (rAAV) virion comprising: i) a variant AAV capsid polypeptide of the present disclosure; and ii) a heterologous nucleic acid comprising a nucleotide sequence encoding a heterologous polypeptide (i.e., a non-AAV polypeptide).

In some cases, an rAAV virion of the present disclosure comprises a capsid protein comprising an amino acid sequence having at least about 85%, at least about 90%, at least about 95%, at least about 98%, or at least about 99%, amino acid sequence identity to the amino acid sequence provided in SEQ ID NO:1; and an insertion of from about 7 amino acids to about 20 amino acids (e.g., 7, 8, 9, 10, 11, 12, 13, 14, 15, 16, 17, 18, 19, or 20 amino acids; e.g., 9 amino acids, 10 amino acids, 11 amino acids, or 12 amino acids) in the GH loop or loop IV relative to a corresponding parental AAV capsid protein. In some embodiments, a subject rAAV virion comprises a capsid protein comprising an amino acid sequence having at least about 85%, at least about 90%, at least about 95%, at least about 98%, or at least about 99%, amino acid sequence identity to the amino acid sequence provided in SEQ ID NO:1; and an insertion of from about 7 amino acids to about 20 amino acids (e.g., 7, 8, 9, 10, 11, 12, 13, 14, 15, 16, 17, 18, 19, or 20 amino acids; e.g., 9 amino acids, 10 amino acids, 11 amino acids, or 12 amino acids) between amino acids 575 and 577 relative to the amino acid sequence provided in SEQ ID NO:1, or at a corresponding site relative to a corresponding parental AAV capsid protein.

In some embodiments, a subject rAAV virion comprises a capsid protein that includes a GH loop comprising an amino acid sequence having at least about 85%, at least about 90%, at least about 95%, at least about 98%, at least about 99%, or 100%, amino acid sequence identity to an amino acid sequence set forth in FIG. 8, and comprising an insertion of from about 7 amino acids to about 20 amino acids (e.g., 7, 8, 9, 10, 11, 12, 13, 14, 15, 16, 17, 18, 19, or 20 amino acids; e.g., 9 amino acids, 10 amino acids, 11 amino acids, or 12 amino acids) between the bolded and underlined amino acids.

An rAAV virion of the present disclosure exhibits at least 5-fold, at least 10-fold, at least 15-fold, at least 20-fold, at least 25-fold, at least 50-fold, or more than 50-fold, increased infectivity of a CNS cell, compared to the infectivity of the CNS cell by a control rAAV virion comprising the corresponding parental AAV capsid protein.

Whether a given rAAV virion exhibits increased infectivity of a CNS cell can be determined by detecting expression in a CNS cell of a heterologous gene product encoded by the rAAV virion, following administration of the rAAV virion. The CNS cell may be in the brain or the spinal cord. For example, an rAAV virion of the present disclosure that comprises: a) a variant capsid of the present disclosure comprising a peptide insert and/or a peptide replacement, as described above; and b) a heterologous nucleotide sequence encoding a heterologous gene product, when administered to a subject, results in a level of the heterologous gene product in a CNS cell, that is at least 2-fold, at least 5-fold, at least 10-fold, at least 15-fold, at least 20-fold, at least 25-fold, at least 50-fold, or more than 50-fold, greater than the level of the gene product in the CNS cell that results when a control rAAV virion that comprises: a) a control AAV capsid that does not comprises the peptide insert and/or the peptide replacement; and b) heterologous nucleotide sequence encoding the heterologous gene product is administered.

In some cases, a subject rAAV virion exhibits at least 5-fold, at least 10-fold, at least 15-fold, at least 20-fold, at least 25-fold, at least 50-fold, or more than 50-fold, increased infectivity of a CNS cell, such as glia, neuron, or microglia, compared to the infectivity of the CNS cell by an AAV virion comprising the corresponding parental AAV capsid protein.

In some embodiments, a subject rAAV virion comprises a capsid protein comprising an insertion of a heterologous peptide comprising the sequence 1) KVSNAAN (SEQ ID NO:42); 2) VVKQRGD (SEQ ID NO:43); 3) VTNVVRA (SEQ ID NO:12); 4) PGPGNTI (SEQ ID NO:13); or 5) QRIVNEV (SEQ ID NO:14) and exhibits at least 5-fold, at least 10-fold, at least 15-fold, at least 20-fold, at least 25-fold, at least 50-fold, or more than 50-fold, increased infectivity of a glial cell, compared to the infectivity of the glial cell by an AAV virion comprising the corresponding parental AAV capsid protein.

In some embodiments, a subject rAAV virion comprises a capsid protein comprising an insertion of a heterologous peptide comprising the sequence 15) KVTRGDT (SEQ ID NO:10); 16) PADNVKA (SEQ ID NO:44); 17) RDGGTKA (SEQ ID NO:45); 18) VEAVGGNVEAVGGN (SEQ ID NO:22); 19) MSLLPYP (SEQ ID NO:23); 20) YFAIYIF (SEQ ID NO:24); 21) HLSDARP (SEQ ID NO:25); 22) CHERRRV (SEQ ID NO:39); 23) LIASFVQ (SEQ ID NO:26); 24) AGGGGKA (SEQ ID NO:27); 25) KGVQERA (SEQ ID NO: 28); or 26) AVRINPG (SEQ ID NO:29) and exhibits at least 5-fold, at least 10-fold, at least 15-fold, at least 20-fold, at least 25-fold, at least 50-fold, or more than 50-fold, increased infectivity of a microglial cell, compared to the infectivity of the microglial cell by an AAV virion comprising the corresponding parental AAV capsid protein.

In some embodiments, a subject rAAV virion comprises a capsid protein comprising an insertion of a heterologous peptide comprising the sequence 6) KSSKNAT (SEQ ID NO:38); 7) PKSRAGA (SEQ ID NO:40); 8) RARGDGG (SEQ ID NO:41); 9) RSSNYVV (SEQ ID NO:16); 10) AAPIDGE (SEQ ID NO:17); 11) IGPVAAD (SEQ ID NO:18); 12) DGVEDAV (SEQ ID NO: 19); 13) GFTDDAT (SEQ ID NO:20); or 14) LEAVATV (SEQ ID NO:21) and exhibits at least 5-fold, at least 10-fold, at least 15-fold, at least 20-fold, at least 25-fold, at least 50-fold, or more than 50-fold, increased infectivity of a neural cell, compared to the infectivity of the neural cell by an AAV virion comprising the corresponding parental AAV capsid protein.

The glial cell, microglial cell, or neuron may be present in the brain of a subject or the spinal cord of the subject. The subject may be a mammal, e.g., rodent, canine, non-human primate, or human. The human subject may be a fetus, neonate, a child, a teen, or an adult.

### Gene Products

An rAAV virion of the present disclosure comprises a heterologous nucleic acid comprising a nucleotide sequence encoding one or more gene products (one or more heterologous gene products). In some cases, the gene product is a polypeptide. In some cases, the gene product is an RNA. In some cases, an rAAV virion of the present disclosure comprises a heterologous nucleotide sequence encoding both a heterologous nucleic acid gene product and a heterologous polypeptide gene product. Where the gene product is an RNA, in some cases, the RNA gene product encodes a polypeptide. Where the gene product is an RNA, in some cases, the RNA gene product does not encode a polypeptide. In some cases, an rAAV virion of the present disclosure comprises a single heterologous nucleic acid comprising a nucleotide sequence encoding a single heterologous gene product. In some cases, an rAAV virion of the present disclosure comprises a single heterologous nucleic acid comprising a nucleotide sequence encoding two heterologous gene products. Where the single heterologous nucleic acid encodes two heterologous gene products, in some cases, nucleotide sequences encoding the two heterologous gene products are operably linked to the same promoter. Where the single heterologous nucleic acid encodes two heterologous gene products, in some cases, nucleotide sequences encoding the two heterologous gene products are operably linked to two different promoters. In some cases, an rAAV virion of the present disclosure comprises a single heterologous nucleic acid comprising a nucleotide sequence encoding three heterologous gene products. Where the single heterologous nucleic acid encodes three heterologous gene products, in some cases, nucleotide sequences encoding the three heterologous gene products are operably linked to the same promoter. Where the single heterologous nucleic acid encodes three heterologous gene products, in some cases, nucleotide sequences encoding the three heterologous gene products are operably linked to two or three different promoters. In some cases, an rAAV virion of the present disclosure comprises two heterologous nucleic acids, each comprising a nucleotide sequence encoding a heterologous gene product.

In some cases, the gene product is a polypeptide-encoding RNA. In some cases, the gene product is an interfering RNA. In some cases, the gene product is an aptamer. In some cases, the gene product is a polypeptide. In some cases, the gene product is a therapeutic polypeptide, e.g., a polypeptide that provides clinical benefit. In some embodiments, the gene product is a site-specific nuclease that provide for site-specific knock-down of gene function. In some embodiments, the gene product is an RNA-guided endonuclease that provides for modification of a target nucleic acid. In some cases, the gene products are: i) an RNA-guided endonuclease that provides for modification of a target nucleic acid; and ii) a guide RNA that comprises a first segment that binds to a target sequence in a target nucleic acid and a second segment that binds to the RNA-guided endonuclease. In some cases, the gene products are: i) an RNA-guided endonuclease that provides for modification of a target nucleic acid; ii) a first guide RNA that comprises a first segment that binds to a first target sequence in a target nucleic acid and a second segment that binds to the RNA-guided endonuclease; and iii) a first guide RNA that comprises a first segment that binds to a second target sequence in the target nucleic acid and a second segment that binds to the RNA-guided endonuclease.

### Interfering RNA

Where the gene product is an interfering RNA (RNAi), suitable RNAi include RNAi that decrease the level of an apoptotic or angiogenic factor in a cell. For example, an RNAi can be an shRNA or siRNA that reduces the level of a gene product that induces or promotes apoptosis in a cell. Genes whose gene products induce or promote apoptosis are referred to herein as “pro-apoptotic genes” and the products of those genes (mRNA; protein) are referred to as “pro-apoptotic gene products.” Pro-apoptotic gene products include, e.g., Bax, Bid, Bak, and Bad gene products. See, e.g., U.S. Pat. No. 7,846,730. siRNA against BACE1 may be expressed in a CNS cell for example to treat Alzheimer's disease. The siRNA may be expressed in neurons, glial, or microglia.

### Polypeptides

Where the gene product is a polypeptide, in some cases, the polypeptide is a polypeptide that enhances function of a CNS cell, e.g., the function of a glial cell, neuron, or microglial cell. Exemplary polypeptides include neuroprotective polypeptides (e.g., glial cell derived neurotrophic factor (GDNF), ciliary neurotrophic factor (CNTF), neurotrophin-4 (NT4), nerve growth factor (NGF), and neurturin (NTN)); anti-angiogenic polypeptides (e.g., a soluble VEGF receptor; a VEGF-binding antibody; a VEGF-binding antibody fragment (e.g., a single chain anti-VEGF antibody); endostatin; tumstatin; angiostatin; a soluble Flt polypeptide (Lai et al. (2005) Mol. Ther. 12:659); an Fc fusion protein comprising a soluble Flt polypeptide (see, e.g., Pechan et al. (2009) Gene Ther. 16:10); pigment epithelium-derived factor (PEDF); a soluble Tie-2 receptor; etc.); tissue inhibitor of metalloproteinases-3 (TIMP-3); a light-responsive opsin, e.g., a rhodopsin; anti-apoptotic polypeptides (e.g., Bcl-2, Bcl-Xl; XIAP); and the like. Suitable polypeptides include, but are not limited to, glial derived neurotrophic factor (GDNF); fibroblast growth factor; fibroblast growth factor 2; neurturin (NTN); ciliary neurotrophic factor (CNTF); nerve growth factor (NGF); neurotrophin-4 (NT4); brain derived neurotrophic factor (BDNF); epidermal growth factor; X-linked inhibitor of apoptosis; survival motor neuron protein (SMN), a X-linked inhibitor of apoptosis, Wnt inhibitory factor-1 (WIF-1), aromatic L-Amino Acid Decarboxylase (AADC), Gigaxonin, a fluorescent protein, or a luciferase enzyme.

Reporter polypeptides, such as, a fluorescent protein or an enzyme that produces a detectable signal may be used to label CNS cells in vitro or in disease models, in vivo.

### Site-Specific Endonucleases

In some cases, a gene product of interest is a site-specific endonuclease that provide for site-specific knock-down of gene function, e.g., where the endonuclease knocks out an allele associated with a CNS disease. For example, where a dominant allele encodes a defective copy of a gene that, when wild-type, is a CNS structural protein and/or provides for CNS function, a site-specific endonuclease can be targeted to the defective allele and knock out the defective allele. In some cases, a site-specific endonuclease is an RNA-guided endonuclease.

In addition to knocking out a defective allele, a site-specific nuclease can also be used to stimulate homologous recombination with a donor DNA that encodes a functional copy of the protein encoded by the defective allele. Thus, e.g., a subject rAAV virion can be used to deliver both a site-specific endonuclease that knocks out a defective allele, and can be used to deliver a functional copy of the defective allele, resulting in repair of the defective allele, thereby providing for production of a functional protein. In some embodiments, a subject rAAV virion comprises a heterologous nucleotide sequence that encodes a site-specific endonuclease; and a heterologous nucleotide sequence that encodes a functional copy of a defective allele, where the functional copy encodes a functional protein. Functional proteins include, e.g., Gigaxonin, and the like.

Site-specific endonucleases that are suitable for use include, e.g., zinc finger nucleases (ZFNs); meganucleases; and transcription activator-like effector nucleases (TALENs), where such site-specific endonucleases are non-naturally occurring and are modified to target a specific gene. Such site-specific nucleases can be engineered to cut specific locations within a genome, and non-homologous end joining can then repair the break while inserting or deleting several nucleotides. Such site-specific endonucleases (also referred to as “INDELs”) then throw the protein out of frame and effectively knock out the gene. See, e.g., U.S. Patent Publication No. 2011/0301073. Suitable site-specific endonucleases include engineered meganucleases and re-engineered homing endonucleases. Suitable endonucleases include an I-Tev1 nuclease. Suitable meganucleases include I-Sce1 (see, e.g., Bellaiche et al. (1999) Genetics 152:1037); and I-Cre1 (see, e.g., Heath et al. (1997) Nature Structural Biology 4:468).

### RNA-Guided Endonucleases

In some cases, the gene product is an RNA-guided endonuclease. In some cases, the gene product is an RNA comprising a nucleotide sequence encoding an RNA-guided endonuclease. In some cases, the gene product is a guide RNA, e.g., a single-guide RNA. In some cases, the gene products are: 1) a guide RNA; and 2) an RNA-guided endonuclease. The guide RNA can comprise: a) a protein-binding region that binds to the RNA-guided endonuclease; and b) a region that binds to a target nucleic acid. An RNA-guided endonuclease is also referred to herein as a “genome editing nuclease.”

Examples of suitable genome editing nucleases are CRISPR/Cas endonucleases (e.g., class 2 CRISPR/Cas endonucleases such as a type II, type V, or type VI CRISPR/Cas endonucleases). A suitable genome editing nuclease is a CRISPR/Cas endonuclease (e.g., a class 2 CRISPR/Cas endonuclease such as a type II, type V, or type VI CRISPR/Cas endonuclease). In some cases, a genome targeting composition includes a class 2 CRISPR/Cas endonuclease. In some cases, a genome targeting composition includes a class 2 type II CRISPR/Cas endonuclease (e.g., a Cas9 protein). In some cases, a genome targeting composition includes a class 2 type V CRISPR/Cas endonuclease (e.g., a Cpf1 protein, a C2c1 protein, or a C2c3 protein). In some cases, a genome targeting composition includes a class 2 type VI CRISPR/Cas endonuclease (e.g., a C2c2 protein; also referred to as a “Cas13a” protein). Also suitable for use is a CasX protein. Also suitable for use is a CasY protein.

In some cases, a genome editing nuclease is a fusion protein that is fused to a heterologous polypeptide (also referred to as a “fusion partner”). In some cases, a genome editing nuclease is fused to an amino acid sequence (a fusion partner) that provides for subcellular localization, i.e., the fusion partner is a subcellular localization sequence (e.g., one or more nuclear localization signals (NLSs) for targeting to the nucleus, two or more NLSs, three or more NLSs, etc.).

In some cases, the genome-editing endonuclease is a Type II CRISPR/Cas endonuclease. In some cases, the genome-editing endonuclease is a Cas9 polypeptide. The Cas9 protein is guided to a target site (e.g., stabilized at a target site) within a target nucleic acid sequence (e.g., a chromosomal sequence or an extrachromosomal sequence, e.g., an episomal sequence, a minicircle sequence, a mitochondrial sequence, a chloroplast sequence, etc.) by virtue of its association with the protein-binding segment of the Cas9 guide RNA. In some cases, the Cas9 polypeptide used in a composition or method of the present disclosure is a Staphylococcus aureus Cas9 (saCas9) polypeptide.

In some cases, a suitable Cas9 polypeptide is a high-fidelity (HF) Cas9 polypeptide. Kleinstiver et al. (2016) Nature 529:490. In some cases, a suitable Cas9 polypeptide exhibits altered PAM specificity. See, e.g., Kleinstiver et al. (2015) Nature 523:481.

In some cases, the genome-editing endonuclease is a type V CRISPR/Cas endonuclease. In some cases, a type V CRISPR/Cas endonuclease is a Cpf1 protein. In some cases, the genome-editing endonuclease is a CasX or a CasY polypeptide. CasX and CasY polypeptides are described in Burstein et al. (2017) Nature 542:237.

### Enzymatically Inactive RNA-Guided Endonucleases

Also suitable for use is an RNA-guided endonuclease with reduced enzymatic activity. Such an RNA-guided endonuclease is referred to as a “dead” RNA-guided endonuclease; for example, a Cas9 polypeptide that comprises certain amino acid substitutions such that it exhibits substantially no endonuclease activity, but such that it still binds to a target nucleic acid when complexed with a guide RNA, is referred to as a “dead” Cas9 or “dCas9.” In some cases, a “dead” Cas9 protein has a reduced ability to cleave both the complementary and the non-complementary strands of a double stranded target nucleic acid. For example, a “nuclease defective” Cas9 lacks a functioning RuvC domain (i.e., does not cleave the non-complementary strand of a double stranded target DNA) and lacks a functioning HNH domain (i.e., does not cleave the complementary strand of a double stranded target DNA). As a non-limiting example, in some cases, the nuclease defective Cas9 protein harbors mutations at amino acid positions corresponding to residues D10 and H840 (e.g., D10A and H840A) of SEQ ID NO: 15 (or the corresponding residues of a homolog of Cas9) such that the polypeptide has a reduced ability to cleave (e.g., does not cleave) both the complementary and the non-complementary strands of a target nucleic acid. Such a Cas9 protein has a reduced ability to cleave a target nucleic acid (e.g., a single stranded or double stranded target nucleic acid) but retains the ability to bind a target nucleic acid. A Cas9 protein that cannot cleave target nucleic acid (e.g., due to one or more mutations, e.g., in the catalytic domains of the RuvC and HNH domains) is referred to as a “nuclease defective Cas9”, “dead Cas9” or simply “dCas9.” Other residues can be mutated to achieve the above effects (i.e. inactivate one or the other nuclease portions). As non-limiting examples, residues D10, G12, G17, E762, H840, N854, N863, H982, H983, A984, D986, and/or A987 of Streptococcus pyogenes Cas9 (or the corresponding amino acids of a Cas9 homolog) can be altered (i.e., substituted). In some cases, two or more of D10, E762, H840, N854, N863, and D986 of Streptococcus pyogenes Cas9 (or the corresponding amino acids of a Cas9 homolog) are substituted. In some cases, D10 and N863 of Streptococcus pyogenes Cas9 (or the corresponding amino acids of a Cas9 homolog) are substituted with Ala. Also, mutations other than alanine substitutions are suitable.

In some cases, the genome-editing endonuclease is an RNA-guided endonuclease (and it corresponding guide RNA) known as Cas9-synergistic activation mediator (Cas9-SAM). The RNA-guided endonuclease (e.g., Cas9) of the Cas9-SAM system is a “dead” Cas9 fused to a transcriptional activation domain (wherein suitable transcriptional activation domains include, e.g., VP64, p65, MyoD1, HSF1, RTA, and SET7/9) or a transcriptional repressor domain (where suitable transcriptional repressor domains include, e.g., a KRAB domain, a NuE domain, an NcoR domain, a SID domain, and a SID4X domain). The guide RNA of the Cas9-SAM system comprises a loop that binds an adapter protein fused to a transcriptional activator domain (e.g., VP64, p65, MyoD1, HSF1, RTA, or SET7/9) or a transcriptional repressor domain (e.g., a KRAB domain, a NuE domain, an NcoR domain, a SID domain, or a SID4X domain). For example, in some cases, the guide RNA is a single-guide RNA comprising an MS2 RNA aptamer inserted into one or two loops of the sgRNA; the dCas9 is a fusion polypeptide comprising dCas9 fused to VP64; and the adaptor/functional protein is a fusion polypeptide comprising: i) MS2; ii) p65; and iii) HSF1. See, e.g., U.S. Patent Publication No. 2016/0355797.

Also suitable for use is a chimeric polypeptide comprising: a) a dead RNA-guided endonuclease; and b) a heterologous fusion polypeptide. Examples of suitable heterologous fusion polypeptides include a polypeptide having, e.g., methylase activity, demethylase activity, transcription activation activity, transcription repression activity, transcription release factor activity, histone modification activity, RNA cleavage activity, DNA cleavage activity, DNA integration activity, or nucleic acid binding activity.

### Guide RNA

A nucleic acid that binds to a class 2 CRISPR/Cas endonuclease (e.g., a Cas9 protein; a type V or type VI CRISPR/Cas protein; a Cpf1 protein; etc.) and targets the complex to a specific location within a target nucleic acid is referred to herein as a “guide RNA” or “CRISPR/Cas guide nucleic acid” or “CRISPR/Cas guide RNA.” A guide RNA provides target specificity to the complex (the RNP complex) by including a targeting segment, which includes a guide sequence (also referred to herein as a targeting sequence), which is a nucleotide sequence that is complementary to a sequence of a target nucleic acid.

In some cases, a guide RNA includes two separate nucleic acid molecules: an “activator” and a “targeter” and is referred to herein as a “dual guide RNA”, a “double-molecule guide RNA”, a “two-molecule guide RNA”, or a “dgRNA.” In some cases, the guide RNA is one molecule (e.g., for some class 2 CRISPR/Cas proteins, the corresponding guide RNA is a single molecule; and in some cases, an activator and targeter are covalently linked to one another, e.g., via intervening nucleotides), and the guide RNA is referred to as a “single guide RNA”, a “single-molecule guide RNA,” a “one-molecule guide RNA”, or simply “sgRNA.”

Where the gene product is an RNA-guided endonuclease, or is both an RNA-guided endonuclease and a guide RNA, the gene product can modify a target nucleic acid. In some cases, e.g., where a target nucleic acid comprises a deleterious mutation in a defective allele (e.g., a deleterious mutation in a CNS cell target nucleic acid), the RNA-guided endonuclease/guide RNA complex, together with a donor nucleic acid comprising a nucleotide sequence that corrects the deleterious mutation (e.g., a donor nucleic acid comprising a nucleotide sequence that encodes a functional copy of the protein encoded by the defective allele), can be used to correct the deleterious mutation, e.g., via homology-directed repair (HDR).

In some cases, the gene products are an RNA-guided endonuclease and 2 separate sgRNAs, where the 2 separate sgRNAs provide for deletion of a target nucleic acid via non-homologous end joining (NHEJ).

In some cases, the gene products are: i) an RNA-guided endonuclease; and ii) one guide RNA. In some cases, the guide RNA is a single-molecule (or “single guide”) guide RNA (an “sgRNA”). In some cases, the guide RNA is a dual-molecule (or “dual-guide”) guide RNA (“dgRNA”).

In some cases, the gene products are: i) an RNA-guided endonuclease; and ii) 2 separate sgRNAs, where the 2 separate sgRNAs provide for deletion of a target nucleic acid via non-homologous end joining (NHEJ). In some cases, the guide RNAs are sgRNAs. In some cases, the guide RNAs are dgRNAs.

In some cases, the gene products are: i) a Cpf1 polypeptide; and ii) a guide RNA precursor; in these cases, the precursor can be cleaved by the Cpf1 polypeptide to generate 2 or more guide RNAs.

The present disclosure provides a method of modifying a target nucleic acid in a CNS cell in an individual, where the target nucleic acid comprises a deleterious mutation, the method comprising administering to the individual an rAAV virion of the present disclosure, where the rAAV virion comprises a heterologous nucleic acid comprising: i) a nucleotide sequence encoding an RNA-guided endonuclease (e.g., a Cas9 endonuclease); ii) a nucleotide sequence encoding a sgRNA that comprises a nucleotide sequence that is complementary to the target nucleic acid; and iii) a nucleotide sequence encoding a donor DNA template that comprises a nucleotide sequence that corrects the deleterious mutation. Administration of the rAAV virion results in correction of the deleterious mutation in the target nucleic acid by HDR.

The present disclosure provides a method of modifying a target nucleic acid in a CNS cell in an individual, where the target nucleic acid comprises a deleterious mutation, the method comprising administering to the individual an rAAV virion of the present disclosure, where the rAAV virion comprises a heterologous nucleic acid comprising: i) a nucleotide sequence encoding an RNA-guided endonuclease (e.g., a Cas9 endonuclease); ii) a nucleotide sequence encoding a first sgRNA that comprises a nucleotide sequence that is complementary to a first sequence in the target nucleic acid; and iii) a nucleotide sequence encoding a second sgRNA that comprises a nucleotide sequence that is complementary to a second sequence in the target nucleic acid. Administration of the rAAV virion results in excision of the deleterious mutation in the target nucleic acid by NHEJ.

### Regulatory Sequences

In some cases, a nucleotide sequence encoding a gene product of interest is operably linked to a transcriptional control element. For example, in some cases, a nucleotide sequence encoding a gene product of interest is operably linked to a constitutive promoter. Promoters which drive or promote expression in most tissues include, but are not limited to, human elongation factor 1α-subunit (EF1α), cytomegalovirus (CMV) immediate-early enhancer and/or promoter, chicken β-actin (CBA) and its derivative CAG, β glucuronidase (GUSB), or ubiquitin C (UBC).

In other cases, a nucleotide sequence encoding a gene product of interest is operably linked to an inducible promoter. In some instances, a nucleotide sequence encoding a gene product of interest is operably linked to a tissue-specific or cell type-specific regulatory element. For example, in some instances, a nucleotide sequence encoding a gene product of interest is operably linked to a CNS cell-specific promoter. For example, in some instances, a nucleotide sequence encoding a gene product of interest is operably linked to a regulatory element that confers selective expression of the operably linked gene in a CNS cell. Non-limiting examples of tissue-specific expression elements for neurons include neuron-specific enolase (NSE), platelet-derived growth factor (PDGF), platelet-derived growth factor B-chain (PDGF-β), synapsin (Syn), methyl-CpG binding protein 2 (MeCP2), Ca²⁺/calmodulin-dependent protein kinase II (CaMKII), metabotropic glutamate receptor 2 (mGluR2), neurofilament light (NFL) or heavy (NFI), β-globin minigene nβ2 preproenkephalin (PPE), enkephalin (Enk) and excitatory amino acid transporter 2 (EAAT2) promoters. Non-limiting examples of tissue-specific expression elements for astrocytes include glial fibrillary acidic protein (GFAP) and EAAT2 promoters. A non-limiting example of a tissue-specific expression element for oligodendrocytes includes the myelin basic protein (MBP) promoter.

### Pharmaceutical Compositions

The present disclosure provides a pharmaceutical composition comprising: a) a subject rAAV virion, as described above; and b) a pharmaceutically acceptable carrier, diluent, excipient, or buffer. In some embodiments, the pharmaceutically acceptable carrier, diluent, excipient, or buffer is suitable for use in a subject, such as, a mouse, rat, cat, dog, horse, primate, or a human.

Such excipients, carriers, diluents, and buffers include any pharmaceutical agent that can be administered without undue toxicity. Pharmaceutically acceptable excipients include, but are not limited to, liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. A wide variety of pharmaceutically acceptable excipients are known in the art and need not be discussed in detail herein. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

### Methods of Delivering a Gene Product to a CNS Cell and Treatment Methods

The present disclosure provides a method of delivering a gene product to a CNS cell in a subject, the method comprising administering to the subject a rAAV as described herein. The gene product can be a polypeptide or an interfering RNA (e.g., an shRNA, an siRNA, and the like), an aptamer, or a site-specific endonuclease (e.g., an RNA-guided endonuclease), as described herein. Delivering a gene product to a CNS cell can provide for treatment of a CNS disease. The CNS cell can be a glia, neuron, or microglia.

The present disclosure provides a method modifying a target nucleic acid in a CNS cell, the method comprising contacting the CNS cell with: 1) an rAAV virion of the present disclosure, wherein the rAAV virion comprises a heterologous nucleic acid comprising a nucleotide sequence encoding an RNA-guided endonuclease that binds a guide RNA; and 2) the guide RNA. The present disclosure provides a method modifying a target nucleic acid in a CNS cell, the method comprising contacting the CNS cell with an rAAV virion of the present disclosure, wherein the rAAV virion comprises a heterologous nucleic acid comprising a nucleotide sequence encoding: i) an RNA-guided endonuclease that binds a guide RNA; and ii) the guide RNA. In some cases, the method comprises contacting the CNS cell with a donor DNA template. In some cases, the RNA-guided endonuclease is a Cas9 polypeptide. In some cases, the guide RNA is a single-guide RNA.

The present disclosure provides a method of treating a CNS disease (e.g., a brain disease or a spinal cord disease), the method comprising administering to an individual in need thereof an effective amount of a subject rAAV virion as described above. A subject rAAV virion can be administered locally at a location in the CNS by, e.g., an injection, or by any other convenient mode or route of administration.

In certain embodiments, the route of administering the rAAV may be subpial, parenchymal, intracranial, intracerebral, spinal, intracerebroventricular, intrathecal or intraventricular.

In certain embodiments, the administering may include direct basal forebrain injection, intraputaminal injection, injection into the subthalamic nucleus, intracerebral injection, intrathecal injection, intraparenchymal injection, or intracranial injection.

A “therapeutically effective amount” will fall in a relatively broad range that can be determined through experimentation and/or clinical trials. For example, for in vivo injection, i.e., injection directly into the CNS, a therapeutically effective dose will be on the order of from about 10⁶ to about 10¹⁵ of the rAAV virions, e.g., from about 10⁸ to 10¹² rAAV virions. For example, for in vivo injection, a therapeutically effective dose will be on the order of from about 10⁶ viral genomes (vg) to about 10¹⁵ vg of the rAAV virions, e.g., from about 10⁸ vg to 10¹² vg. For in vitro transduction, an effective amount of rAAV virions to be delivered to cells will be on the order of from about 10⁸ to about 10¹³ of the rAAV virions. For example, for in vitro transduction, an effective amount of rAAV virions to be delivered to cells will be on the order of from about 10⁸ to about 10¹³ vg of the rAAV virions. As another example, for in vitro transduction, an effective amount of rAAV virions to be delivered to cells will be on the order of from about 10 vg/cell to about 10⁴ vg/cell. Other effective dosages can be readily established by one of ordinary skill in the art through routine trials establishing dose response curves.

In some embodiments, more than one administration (e.g., two, three, four or more administrations) may be employed to achieve the desired level of gene expression. In some cases, the more than one administration is administered at various intervals, e.g., daily, weekly, twice monthly, monthly, every 3 months, every 6 months, yearly, etc. In some cases, multiple administrations are administered over a period of time of from 1 month to 2 months, from 2 months to 4 months, from 4 months to 8 months, from 8 months to 12 months, from 1 year to 2 years, from 2 years to 5 years, or more than 5 years.

The rAAV disclosed herein find use in treatment of CNS diseases. As used herein, a CNS disease refers to a CNS-related disorder or condition. CNS-related disorder may affect the spinal cord (e.g., a myelopathy), brain (e.g., a encephalopathy) or tissues surrounding the brain and spinal cord. A CNS-related disorder may be of a genetic origin, either inherited or acquired through a somatic mutation. A CNS-related disorder may be a psychological condition or disorder, e.g., Attention Deficient Hyperactivity Disorder, Autism Spectrum Disorder, Mood Disorder, Schizophrenia, Depression, Rett Syndrome, etc. A CNS-related disorder may be an autoimmune disorder. A CNS-related disorder may also be a cancer of the CNS, e.g., brain cancer. A CNS-related disorder that is a cancer may be a primary cancer of the CNS, e.g., an astrocytoma, glioblastomas, etc., or may be a cancer that has metastasized to CNS tissue, e.g., a lung cancer that has metastasized to the brain. A CNS-related disorder may be caused by an injury, e.g., an extraneous injury (e.g., trauma from physical injury) or a stroke. Non-limiting examples of CNS-related disorders, include Parkinson's Disease, Lysosomal Storage Disease, Ischemia, Neuropathic Pain, Amyotrophic lateral sclerosis (ALS), Multiple Sclerosis (MS), Canavan disease (CD), glioblastoma, brain injury (e.g., a stroke) or spinal cord injury (e.g., a contusion), Alzheimer's disease, Multiple Sclerosis, Huntington's disease, Batten disease, giant axonal neuropathy, etc.

The present disclosure provides methods of treating a CNS disease. The methods generally involve administering an rAAV virion of the present disclosure, or a composition comprising an rAAV virion of the present disclosure, to the CNS of an individual in need thereof. Non-limiting methods for assessing treatment of CNS diseases include measuring functional changes, e.g., changes in anatomy using anatomical and/or photographic measures, symptoms, cognitive tasks, or physical tasks.

Assessments can include, but are not limited to ADAS-cog (Alzheimer Disease Assessment Scale—cognitive), MMSE (Mini-Mental State Examination), GDS (Geriatric Depression Scale). FAQ (Functional Activities Questionnaire), ADL (Activities of Daily Living), GPCOG (General Practitioner Assessment of Cognition), Mini-Cog, AMTS (Abbreviated Mental Test Score), Clock-drawing test, 6-CIT (6-item Cognitive Impairment Test), TYM (Test Your Memory), MoCa (Montreal Cognitive Assessment), ACE-R (Addenbrookes Cognitive Assessment), MIS (Memory Impairment Screen), BADLS (Bristol Activities of Daily Living Scale), Barthel Index, Functional Independence Measure, Instrumental Activities of Daily Living, IQCODE (Informant Questionnaire on Cognitive Decline in the Elderly), Neuropsychiatric Inventory, The Cohen-Mansfield Agitation Inventory, BEHAVE-AD, EuroQol, Short Form-36 and/or MBR Caregiver Strain Instrument, or any of the other tests as described in Sheehan B Ther Adv Neurol Disord 5 (6): 349-358 (2012), the contents of which are herein incorporated by reference in their entirety.

In certain embodiments, the CNS disease may be Parkinson's disease and the heterologous nucleic acid delivered to the CNS cells of the subject by the rAAV may encode glial cell derived neurotrophic factor (GDNF).

In certain embodiments, the CNS disease may be Alzheimer's disease and the heterologous nucleic acid delivered to the CNS cells of the subject by the rAAV may encode a siRNA that targets BACE1.

In certain embodiments, the CNS disease may be Giant Axonal Neuropathy and the heterologous nucleic acid delivered to the CNS cells of the subject by the rAAV may encode a Gigaxonin.

Methods for treating Canavan disease (CD) in a subject in need thereof are provided herein. Canavan disease is caused by a defective aspartoacylase (ASPA) gene which is responsible for the production of the enzyme aspartoacylase. This enzyme normally breaks down the concentrated brain molecule N-acetyl aspartate. Decreased aspartoacylase activity in subjects with CD prevents the normal breakdown of N-acetyl aspartate, and the lack of breakdown appears to interfere with growth of the myelin sheath of the nerve fibers in the brain. Symptoms of Canavan disease, which may appear in early infancy and progress rapidly, may include mental retardation, loss of previously acquired motor skills, feeding difficulties, to abnormal muscle tone (i.e., floppiness or stiffness), poor head control, and megalocephaly (abnormally enlarged head). Paralysis, blindness, or seizures may also occur. Aspects of the invention improve one or more symptoms of CD in a subject by administering to the subject a recombinant AAV as provided herein harboring a nucleic acid that expresses aspartoacylase (ASPA).

In certain cases, the rAAV disclosed herein may be used to deliver a defective gene, create a knock-out, create a mutation, and the like in a laboratory animal for generating a disease model which disease affect a CNS cell.

### Nucleic Acids and Host Cells

The present disclosure provides an isolated nucleic acid comprising a nucleotide sequence that encodes a subject variant adeno-associated virus (AAV) capsid protein as described above, where the variant AAV capsid protein comprises an insertion of from about 7 amino acids to about 20 amino acids in the GH loop or loop IV relative to a corresponding parental AAV capsid protein, or where the variant AAV capsid protein comprises a replacement of from about 7 amino acids to about 20 amino acids in the GH loop or loop IV relative to a corresponding parental AAV capsid protein with a heterologous peptide of from about 7 amino acids to about 20 amino acids; and where the variant capsid protein, when present in an AAV virion, provides for increased infectivity of a CNS cell compared to the infectivity of the CNS cell by an AAV virion comprising the corresponding parental AAV capsid protein. A subject isolated nucleic acid can be an AAV vector, e.g., a recombinant AAV vector.

A subject recombinant AAV vector can be used to generate a subject recombinant AAV virion, as described above. Thus, the present disclosure provides a recombinant AAV vector that, when introduced into a suitable cell, can provide for production of a subject recombinant AAV virion.

The present invention further provides host cells, e.g., isolated (genetically modified) host cells, comprising a subject nucleic acid. A subject host cell can be an isolated cell, e.g., a cell in in vitro culture. A subject host cell is useful for producing a subject rAAV virion, as described below. Where a subject host cell is used to produce a subject rAAV virion, it is referred to as a “packaging cell.” In some embodiments, a subject host cell is stably genetically modified with a subject nucleic acid. In other embodiments, a subject host cell is transiently genetically modified with a subject nucleic acid.

A subject nucleic acid is introduced stably or transiently into a host cell, using established techniques, including, but not limited to, electroporation, calcium phosphate precipitation, liposome-mediated transfection, and the like. For stable transformation, a subject nucleic acid will generally further include a selectable marker, e.g., any of several well-known selectable markers such as neomycin resistance, and the like.

A subject host cell is generated by introducing a subject nucleic acid into any of a variety of cells, e.g., mammalian cells, including, e.g., murine cells, and primate cells (e.g., human cells). Suitable mammalian cells include, but are not limited to, primary cells and cell lines, where suitable cell lines include, but are not limited to, 293 cells, 293T cells, COS cells, HeLa cells, Vero cells, 3T3 mouse fibroblasts, C3H10T1/2 fibroblasts, CHO cells, and the like. Non-limiting examples of suitable host cells include, e.g., HeLa cells (e.g., American Type Culture Collection (ATCC) No. CCL-2), CHO cells (e.g., ATCC Nos. CRL9618, CCL61, CRL9096), 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RAT1 cells, mouse L cells (ATCC No. CCLI.3), human embryonic kidney (HEK) cells (ATCC No. CRL1573), HLHepG2 cells, and the like. A subject host cell can also be made using a baculovirus to infect insect cells such as Sf9 cells, which produce AAV (see, e.g., U.S. Pat. No. 7,271,002; U.S. patent application Ser. No. 12/297,958)

In some embodiments, a subject genetically modified host cell includes, in addition to a nucleic acid comprising a nucleotide sequence encoding a variant AAV capsid protein, as described above, a nucleic acid that comprises a nucleotide sequence encoding one or more AAV rep proteins. In other embodiments, a subject host cell further comprises an rAAV vector. An rAAV virion can be generated using a subject host cell. Methods of generating an rAAV virion are described in, e.g., U.S. Patent Publication No. 2005/0053922 and U.S. Patent Publication No. 2009/0202490.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:
1. A recombinant adeno-associated virus (rAAV) comprising:
(a) a variant AAV capsid protein, wherein the variant AAV capsid protein comprises an insertion of a heterologous peptide comprising the amino acid sequence:

```
  1)
(SEQ ID NO: 42)
KVSNAAN;
  2)
(SEQ ID NO: 43)
VVKQRGD;
  3)
(SEQ ID NO: 12)
VTNVVRA;
  4)
(SEQ ID NO: 13)
PGPGNTI;
  5)
(SEQ ID NO: 14)
QRIVNEV;
  6)
(SEQ ID NO: 38)
KSSKNAT;
  7)
(SEQ ID NO: 40)
PKSRAGA;
  8)
(SEQ ID NO: 41)
RARGDGG;
  9)
(SEQ ID NO: 16)
RSSNYVV;
10)
(SEQ ID NO: 17)
AAPIDGE;
11)
(SEQ ID NO: 18)
IGPVAAD;
12)
(SEQ ID NO: 19)
DGVEDAV;
13)
(SEQ ID NO: 20)
GFTDDAT;
14)
(SEQ ID NO: 21)
LEAVATV;
15)
(SEQ ID NO: 10)
KVTRGDT;
16)
(SEQ ID NO: 44)
PADNVKA;
17)
(SEQ ID NO: 45)
RDGGTKA;
18)
(SEQ ID NO: 22)
VEAVGGNVEAVGGN;
19)
(SEQ ID NO: 23)
MSLLPYP;
20)
(SEQ ID NO: 24)
YFAIYIF;
21)
(SEQ ID NO: 25)
HLSDARP;
22)
(SEQ ID NO: 39)
CHERRRV;
23)
(SEQ ID NO: 26)
LIASFVQ;
24)
(SEQ ID NO: 27)
AGGGGKA;
25)
(SEQ ID NO: 28)
KGVQERA;
or
26)
(SEQ ID NO: 29)
AVRINPG.
```

and
(b) a heterologous nucleic acid comprising a nucleotide sequence encoding a gene product.
2. The rAAV of aspect 1, wherein the rAAV comprises the heterologous peptide comprising the amino acid sequence:

1)
(SEQ ID NO: 42)
KVSNAAN;
2)
(SEQ ID NO: 43)
VVKQRGD;
3)
(SEQ ID NO: 12)
VTNVVRA;
4)
(SEQ ID NO: 13)
PGPGNTI;
or
5)
(SEQ ID NO: 14)
QRIVNEV,

and wherein the rAAV exhibits increased infectivity of a glial cell compared to the infectivity of the glial cell by a control rAAV comprising the corresponding parental AAV capsid protein.
3. The rAAV of aspect 1 or 2, wherein the rAAV exhibits at least 2-fold, at least 3-fold, at least 5-fold, or at least 10-fold increased infectivity of a glial cell compared to the infectivity of the glial cell by a control rAAV comprising the corresponding parental AAV capsid protein.
4. The rAAV of aspect 1, wherein the rAAV comprises the heterologous peptide comprising the amino acid sequence:

```
  6)
(SEQ ID NO: 38)
KSSKNAT;
  7)
(SEQ ID NO: 40)
PKSRAGA;
  8)
(SEQ ID NO: 41)
RARGDGG;
  9)
(SEQ ID NO: 16)
RSSNYVV;
10)
(SEQ ID NO: 17)
AAPIDGE;
11)
(SEQ ID NO: 18)
IGPVAAD;
12)
(SEQ ID NO: 19)
DGVEDAV;
13)
(SEQ ID NO: 20)
GFTDDAT;
or
14)
(SEQ ID NO: 21)
LEAVATV,
```

and wherein the rAAV exhibits increased infectivity of a neuronal cell compared to the infectivity of the neuronal cell by a control rAAV comprising the corresponding parental AAV capsid protein.
5. The rAAV of aspect 1 or 4, wherein the rAAV exhibits at least 2-fold, at least 3-fold, at least 5-fold, or at least 10-fold increased infectivity of a neuronal cell compared to the infectivity of the neuronal cell by a control rAAV comprising the corresponding parental AAV capsid protein.
6. The rAAV of aspect 1, wherein the rAAV comprises the heterologous peptide comprising the amino acid sequence:

15)
(SEQ ID NO: 10)
KVTRGDT;
16)
(SEQ ID NO: 44)
PADNVKA;
17)
(SEQ ID NO: 45)
RDGGTKA;
18)
(SEQ ID NO: 22)
VEAVGGNVEAVGGN;
19)
(SEQ ID NO: 23)
MSLLPYP;
20)
(SEQ ID NO: 24)
YFAIYIF;
21)
(SEQ ID NO: 25)
HLSDARP;
22)
(SEQ ID NO: 39)
CHERRRV;
23)
(SEQ ID NO: 26)
LIASFVQ;
24)
(SEQ ID NO: 27)
AGGGGKA;
25)
(SEQ ID NO: 28)
KGVQERA;
or
26)
(SEQ ID NO: 29)
AVRINPG,

and wherein the rAAV exhibits increased infectivity of a microglial cell compared to the infectivity of the microglial cell by a control rAAV comprising the corresponding parental AAV capsid protein.
7. The rAAV of aspect 1 or 6, wherein the rAAV exhibits at least 2-fold, at least 3-fold, at least 5-fold, or at least 10-fold increased infectivity of a microglial cell compared to the infectivity of the microglial cell by a control rAAV comprising the corresponding parental AAV capsid protein.
8. The rAAV of any one of aspects 1-7, wherein the heterologous peptide is inserted at an insertion site between amino acids corresponding to amino acids 570 and 611 of VP1 of AAV5, or the corresponding position in the capsid protein of another AAV serotype.
9. The rAAV of any one of aspects 1-8, wherein the heterologous peptide is inserted at an insertion site between amino acids corresponding to amino acids 575-577 of VP1 of AAV5, or the corresponding position in the capsid protein of another AAV serotype.
10. The rAAV of any one of aspects 1-10, wherein gene product is a guide RNA, an interfering RNA, a micro RNA, or an aptamer.
11. The rAAV of any one of aspects 1-9, wherein the gene product is a polypeptide.
12. The rAAV of aspect 12, wherein the polypeptide is a neuroprotective polypeptide, a polypeptide that enhances function of a central nervous system cell, an anti-apoptotic polypeptide, an apoptosis-inducing polypeptide, or a reporter polypeptide.
13. The rAAV of aspect 13, wherein the polypeptide is an RNA-guided endonuclease selected from a type II CRISPR/Cas polypeptide, a type V CRISPR/Cas polypeptide, and a type VI CRISPR/Cas polypeptide.
14. The rAAV of aspect 13, wherein the RNA-guided endonuclease is an enzymatically inactive type II CRISPR/Cas polypeptide.
15. The rAAV of aspect 9, wherein the gene product is an RNA-guided endonuclease and a guide RNA.
16. A pharmaceutical composition comprising:
a) a recombinant adeno-associated virus of any one of aspects 1-15; and
b) a pharmaceutically acceptable excipient.
17. A method of delivering a gene product to a central nervous system cell in a subject, the method comprising administering to the subject an adeno-associated virus (rAAV) according any one of aspects 1-15 or the composition of aspect 16.
18. The method of aspect 17, wherein the polypeptide is a glial derived neurotrophic factor, a fibroblast growth factor 2 (FGF2), a nerve growth factor (NGF), a brain derived neurotrophic factor (BDNF), glial cell derived neurotrophic factor (GDNF), survival motor neuron protein (SMN), a X-linked inhibitor of apoptosis, Wnt inhibitory factor-1 (WIF-1), aromatic L-Amino Acid Decarboxylase (AADC), Gigaxonin, a fluorescent protein, or a luciferase enzyme.
19. The method of aspect 17, wherein the polypeptide is an RNA-guided endonuclease.
20. A method of treating a central nervous system disease, the method comprising administering to a subject in need thereof an effective amount of an adeno-associated virus (rAAV) according to any one of aspects 1-15 or the composition of aspect 16.
21. The method of aspect 20, wherein said administering is parenchymal, intracranial, intracerebral, spinal, intracerebroventricular, intrathecal or intraventricular.
22. The method of aspect 20 or 21, wherein the CNS disease is spinal muscular atrophy, Parkinson's disease, spinal cord injury, neuropathic pain, Alzheimer's disease, Multiple Sclerosis, Huntington's disease, Batten disease, giant axonal neuropathy, aromatic-1-amino-acid decarboxylase (AADC) deficiency, or glioblastoma multiforme.
23. An isolated nucleic acid comprising a nucleotide sequence that encodes a variant adeno-associated virus (AAV) capsid protein, wherein the variant AAV capsid protein comprises an insertion of a heterologous peptide comprising the amino acid sequence:

```
  1)
(SEQ ID NO: 42)
KVSNAAN;
  2)
(SEQ ID NO: 43)
VVKQRGD;
  3)
(SEQ ID NO: 12)
VTNVVRA;
  4)
(SEQ ID NO: 13)
PGPGNTI;
  5)
(SEQ ID NO: 14
QRIVNEV;
  6)
(SEQ ID NO: 38)
KSSKNAT;
  7)
(SEQ ID NO: 40)
PKSRAGA;
  8)
(SEQ ID NO: 41)
RARGDGG;
  9)
(SEQ ID NO: 16)
RSSNYVV;
10)
(SEQ ID NO: 17)
AAPIDGE;
11)
(SEQ ID NO: 18)
IGPVAAD;
12)
(SEQ ID NO: 19)
DGVEDAV;
13)
(SEQ ID NO: 20)
GFTDDAT;
14)
(SEQ ID NO: 21)
LEAVATV;
15)
(SEQ ID NO: 10)
KVTRGDT;
16)
(SEQ ID NO: 44)
PADNVKA;
17)
(SEQ ID NO: 45)
RDGGTKA;
18)
(SEQ ID NO: 22)
VEAVGGNVEAVGGN;
19)
(SEQ ID NO: 23)
MSLLPYP;
20)
(SEQ ID NO: 24)
YFAIYIF;
21)
(SEQ ID NO: 25)
HLSDARP;
22)
(SEQ ID NO: 39)
CHERRRV;
23)
(SEQ ID NO: 26)
LIASFVQ;
24)
(SEQ ID NO: 27)
AGGGGKA;
25)
(SEQ ID NO: 28)
KGVQERA;
or
26)
(SEQ ID NO: 29)
AVRINPG.
```

24. The isolated nucleic acid of aspect 23, wherein the variant capsid protein, when present in an recombinant AAV, provides for increased infectivity of a central nervous system (CNS) cell.
25. The isolated nucleic acid of aspect 23 or 24, wherein the variant capsid protein comprises the heterologous peptide comprising the amino acid sequence:

```
  1)
(SEQ ID NO: 42)
KVSNAAN;
  2)
(SEQ ID NO: 43)
VVKQRGD;
  3)
(SEQ ID NO: 12)
VTNVVRA;
  4)
(SEQ ID NO: 13)
PGPGNTI;
or
  5)
(SEQ ID NO: 14
QRIVNEV;
```

and wherein when present in a rAAV, the rAAV exhibits increased infectivity of a glial cell compared to the infectivity of the glial cell by a control rAAV comprising the corresponding parental AAV capsid protein.
26. The isolated nucleic acid of aspect 25, wherein the rAAV exhibits at least 2-fold, at least 3-fold, at least 5-fold, or at least 10-fold increased infectivity of a glial cell compared to the infectivity of the glial cell by a control AAV comprising the corresponding parental rAAV capsid protein.
27. The isolated nucleic acid of aspect 25 or 26, wherein the variant capsid protein comprises the heterologous peptide comprising the amino acid sequence:

6)
(SEQ ID NO: 38)
KSSKNAT;
7)
(SEQ ID NO: 40)
PKSRAGA;
8)
(SEQ ID NO: 41)
RARGDGG;
9)
(SEQ ID NO: 16)
RSSNYVV;
10)
(SEQ ID NO: 17)
AAPIDGE;
11)
(SEQ ID NO: 18)
IGPVAAD;
12)
(SEQ ID NO: 19)
DGVEDAV;
13)
(SEQ ID NO: 20)
GFTDDAT;
or
14)
(SEQ ID NO: 21)
LEAVATV,

and wherein when present in a rAAV, the rAAV exhibits increased infectivity of a neuronal cell compared to the infectivity of the neuronal cell by a control AAV comprising the corresponding parental AAV capsid protein.
28. The isolated nucleic acid of aspect 27, wherein the rAAV exhibits at least 2-fold, at least 3-fold, at least 5-fold, or at least 10-fold increased infectivity of a neuronal cell compared to the infectivity of the neuronal cell by a control AAV comprising the corresponding parental AAV capsid protein.
29. The isolated nucleic acid of aspect 23 or 24, wherein the variant capsid protein comprises the heterologous peptide comprising the amino acid sequence:

15)
(SEQ ID NO: 10)
KVTRGDT;
16)
(SEQ ID NO: 44)
PADNVKA;
17)
(SEQ ID NO: 45)
RDGGTKA;
18)
(SEQ ID NO: 22)
VEAVGGNVEAVGGN;
19)
(SEQ ID NO: 23)
MSLLPYP;
20)
(SEQ ID NO: 24)
YFAIYIF;
21)
(SEQ ID NO: 25)
HLSDARP;
22)
(SEQ ID NO: 39)
CHERRRV;
23)
(SEQ ID NO: 26)
LIASFVQ;
24)
(SEQ ID NO: 27)
AGGGGKA;
25)
(SEQ ID NO: 28)
KGVQERA;
or
26)
(SEQ ID NO: 29)
AVRINPG,

and wherein when present in a rAAV, the rAAV exhibits increased infectivity of a microglial cell compared to the infectivity of the microglial cell by a control rAAV comprising the corresponding parental AAV capsid protein.
30. The isolated nucleic acid of aspect 29, wherein the AAV exhibits at least 2-fold, at least 3-fold, at least 5-fold, or at least 10-fold increased infectivity of a microglial cell compared to the infectivity of the microglial cell by a control AAV comprising the corresponding parental AAV capsid protein.
31. The isolated nucleic acid of any one of aspects 23-30, wherein the heterologous peptide is inserted at an insertion site between amino acids corresponding to amino acids 570 and 611 of VP1 of AAV5, or the corresponding position in the capsid protein of another AAV serotype.
32. The isolated nucleic acid of any one of aspects 23-31, wherein the heterologous peptide is inserted at an insertion site between amino acids corresponding to amino acids 575-577 of VP1 of AAV5, or the corresponding position in the capsid protein of another AAV serotype.
33. An isolated, genetically modified host cell comprising the nucleic acid of any one of aspects 23-32.
34. A variant adeno-associated virus (AAV) capsid protein encoded by the isolated nucleic acid of any one of aspects 23-32.
35. A host cell producing an AAV of any one of aspects 1-15.
36. The host cell of aspect 35, wherein the host cell comprises the nucleic acid of any one of aspects 23-32.
37. The host cell of aspect 35 or 36, further comprising a heterologous nucleic acid comprising a nucleotide sequence encoding a gene product.
38. The host cell of aspect 37, wherein gene product is a guide RNA, an interfering RNA, a micro RNA, or an aptamer.
39. The host cell of aspect 37, wherein the gene product is a polypeptide.
40. The host cell of aspect 39, wherein the polypeptide is a neuroprotective polypeptide, a polypeptide that enhances function of a central nervous system cell, an anti-apoptotic polypeptide, an apoptosis-inducing polypeptide, or a reporter polypeptide.
41. The host cell of aspect 39, wherein the polypeptide is an RNA-guided endonuclease selected from a type II CRISPR/Cas polypeptide, a type V CRISPR/Cas polypeptide, and a type VI CRISPR/Cas polypeptide.
42. The host cell of aspect 41, wherein the RNA-guided endonuclease is an enzymatically inactive type II CRISPR/Cas polypeptide.
43. The host cell of aspect 37, wherein the gene product is an RNA-guided endonuclease and a guide RNA.
44. The host cell of any one of aspects 35-43, wherein the nucleotide sequence encoding a gene product is flanked by AAV inverted terminal repeats.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1: AAV Virions Comprising Variant AAV Capsids

A number of variants of AAV capsids were derived through directed evolution technologies guided by machine-learning (ML) design for developing efficient AAV vectors that target specific CNS cell populations in human brain tissues.

To optimize library design for directed evolution, a ML-based framework was developed to design an AAV5-capsid library, focusing on a 7-amino acid insertion region at position 575-577, the 3-fold symmetry axis that was identified as a retargeting site for cell-specific entry (O. J. Müller, et al., Nat Biotechnol. 21 (9): p. 1040-6 (2003); L. Perabo., et al., Mol Ther. 8 (1): p. 151-7 (2003). By genetically diversifying the 7-amino acid region, a library of AAV5-based variants was generated that can potentially overcome current barriers in human CNS delivery. Using the trained predictive model, a synthetic library was designed that achieves 5-fold higher packaging fitness than the state-of-the-art library (NNK library), with approximately 10-fold more successful variants after primary brain infection. The “NNK” moniker refers to a broadly used strategy (L. Zheng, et al., Nucleic Acids Research. 32 (14): p. e115-e115 (2004), S. Kille, et al., ACS Synthetic Biology. 2 (2): p. 83-92 (2013), A. Li, et al., Applied Microbiology and Biotechnology. 102 (14): p. 6095-6103 (2018) involving a uniform distribution over all four nucleotides (N) in the first two positions of a codon, and equal probability on nucleotides G and T (K) in the third position; where the K in the third position was chosen to reduce the chance of stop codons which typically render the protein non-functional. Each of the 7 amino acids in the insertion is sampled at random from this distribution during library construction. Although NNK libraries are among the most promising AAV libraries (D. Dalkara, et al., Sci Transl Med. 5 (189): p. 189ra76 (2013)), a substantial fraction (>50%) of the variants in these libraries fail to package (i.e., do not assemble into viable capsids, and many more have lower packaging fitness than the parental virus (L. C. Byrne, et al., JCI insight. 5 (10): p. e135112 (2020), K. Adachi, et al. Nature Communications. 5 (1): p. 3075 (2014)). For example, placing a large hydrophobic residue in the 7-mer (solvent-exposed) region is likely destabilizing. Much of the experimental library is thus effectively wasted on poor fitness variants.

The synthetic ML-library was then packaged such that each viral genome was encapsulated within the capsid protein shell that is genome encoded. Therefore, functional improvements identified through selection can be linked to the genome sequence contained within the viral capsid. Specifically, the aforementioned library was transfected into packaging cell lines (HEK 293T cells) to produce viral particles. After purification of viral particles and titer quantifications for library using digital-droplet PCR, a DNA localization-based screening selection process was applied to identify AAV variants with the ability to infect either neuronal or glial cells in the primary human brain tissues.

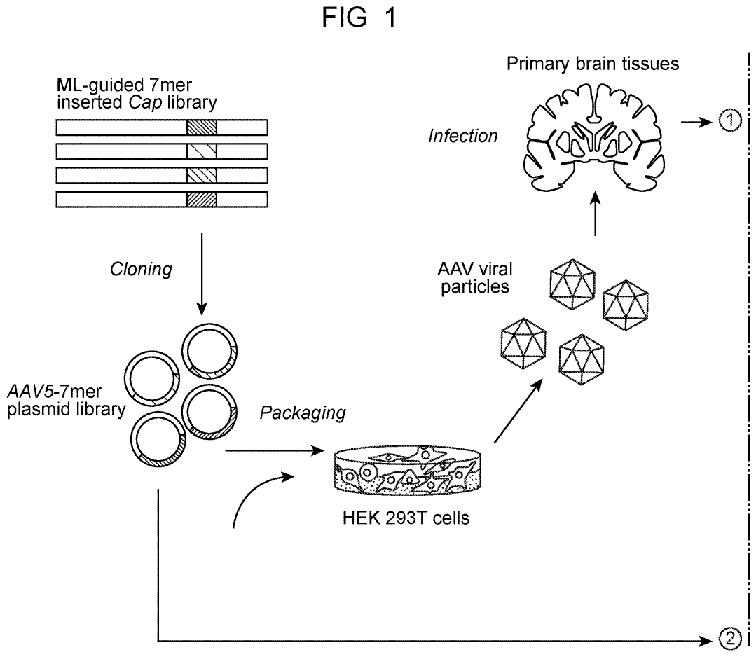

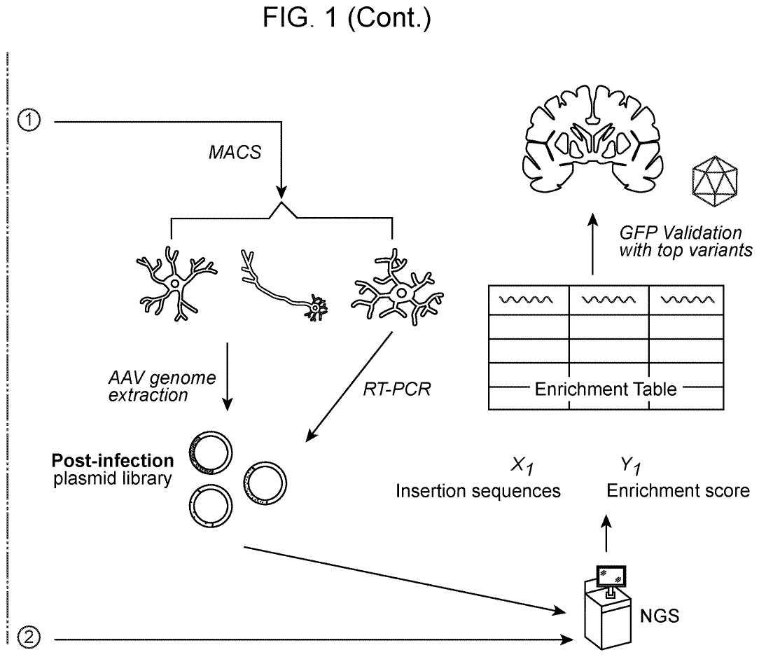

Primary brain samples were harvested from prenatal tissue ranging from gestational week (GW) 19 to 23 and sectioned into 300 μm thickness. Slices were transferred to slice culture inserts on six-well culture plates and cultured in prenatal brain slice culture medium containing 66% (vol/vol) Eagle's basal medium, 25% (vol/vol) HBSS, 2% (vol/vol) B27, 1% N2 supplement, 1% penicillin/streptomycin and GlutaMax in a 37° C. incubator at 5% CO₂, 8% O₂ at the liquid-air interface created by the cell-culture insert. ~50 μL of ~10E12 vg/mL titer AAV library vector were applied directly onto the brain slice. After three days post-infection, brain slices were enzymatically dissociated into single-cell suspension. Functional selective pressure was imposed by harvesting glial- and neuronal-only cells using magnetically-activated cell sorting with PSA-NCAM beads for the neuronal fraction and resulting flow-through depleted of neurons for the glial fraction. Using a Hirt-extraction protocol and PCR-based recovery of cap variants with deep sequencing, highly enriched AAV variants that successfully transduce human glial or neuronal cells specifically were identified (FIG. 1).

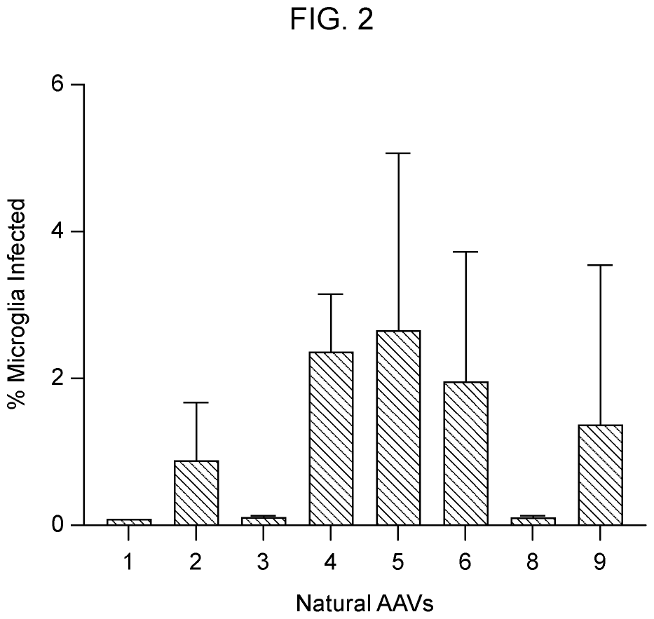

FIG. 2 shows that natural AAV serotypes transduce human microglia in brain slices very poorly. For the microglia population, a transcription-based RNA selection was applied since microglia cells are phagocytes that likely present engulfed DNA from other cells. To generate transcription-competent AAV library for RNA-driven directed evolution, a non-AAV promoter (CMV) was inserted upstream of the cap gene, which would confer cellular expression while retaining the minimal regulatory elements essential for capsid protein expression. For microglia selection, adult human brain slices were used that were sectioned at 300 μm thickness and maintained in culture in air-liquid interface. After library infection, slices were cultured for five days to enable efficient viral transcript accumulation. After microglia cell sorting with MACS beads against CD11b, the whole RNA transcripts were extracted from cells, and capsid library sequences were recovered by RT-PCR. Amplified pools were then deep sequenced to identify highly enriched AAV sequences that achieved high transcription and transduction in the CNS microglia population only.

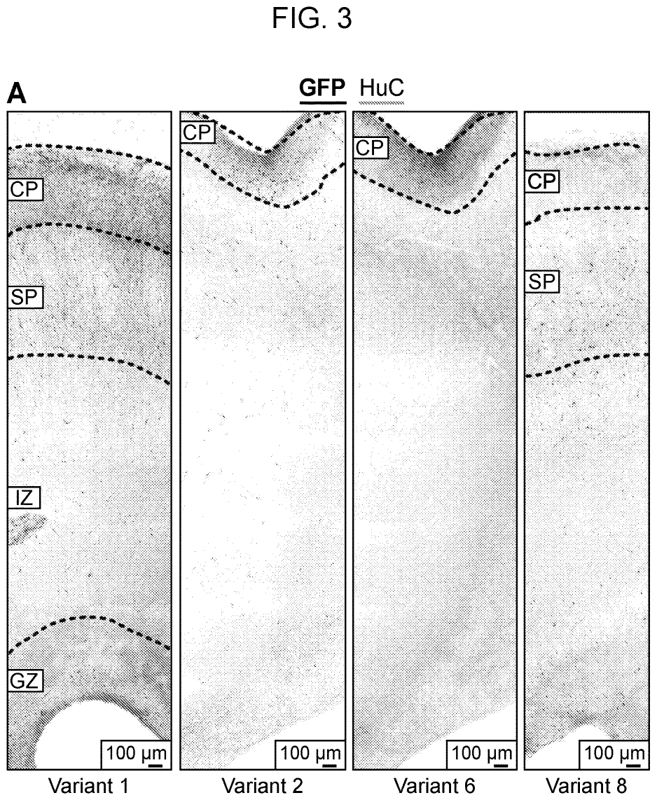

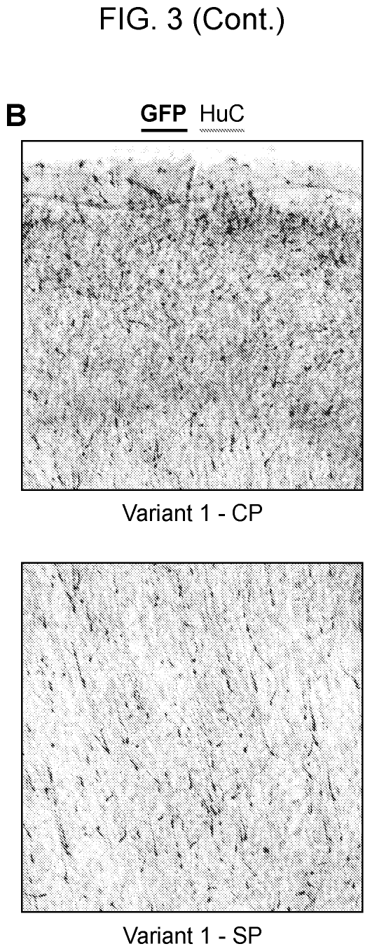

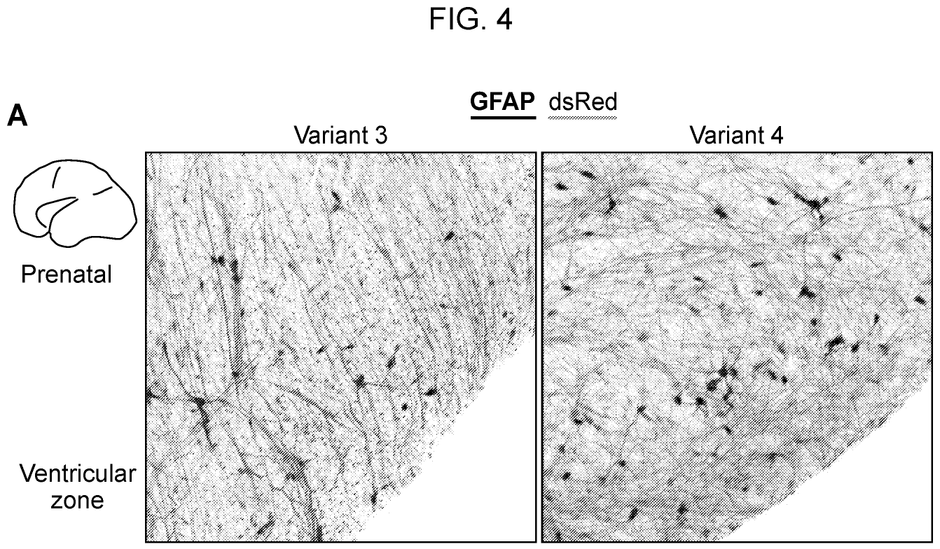

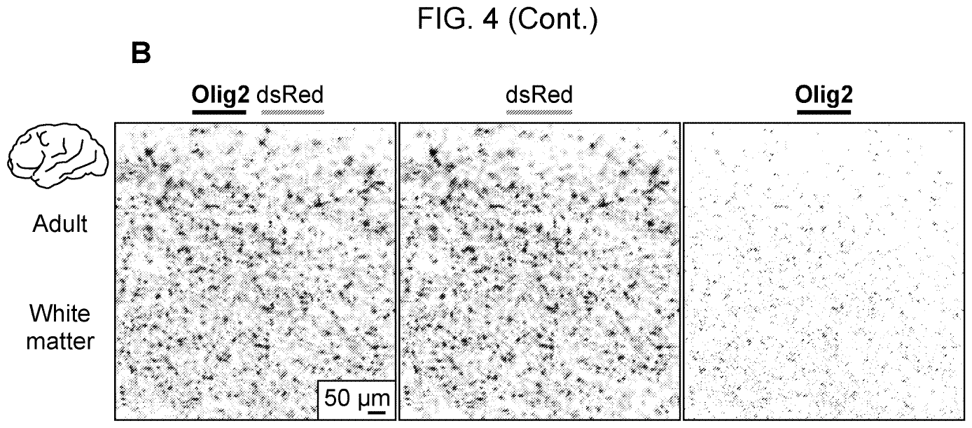

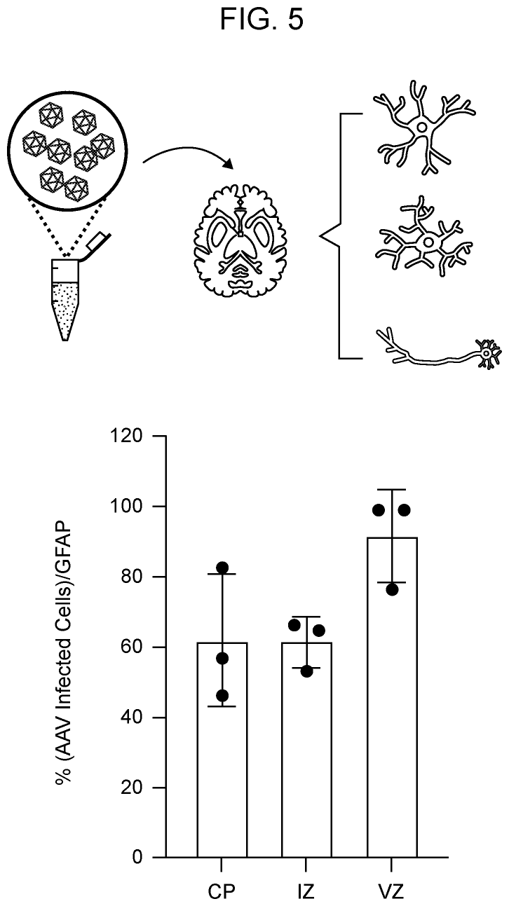

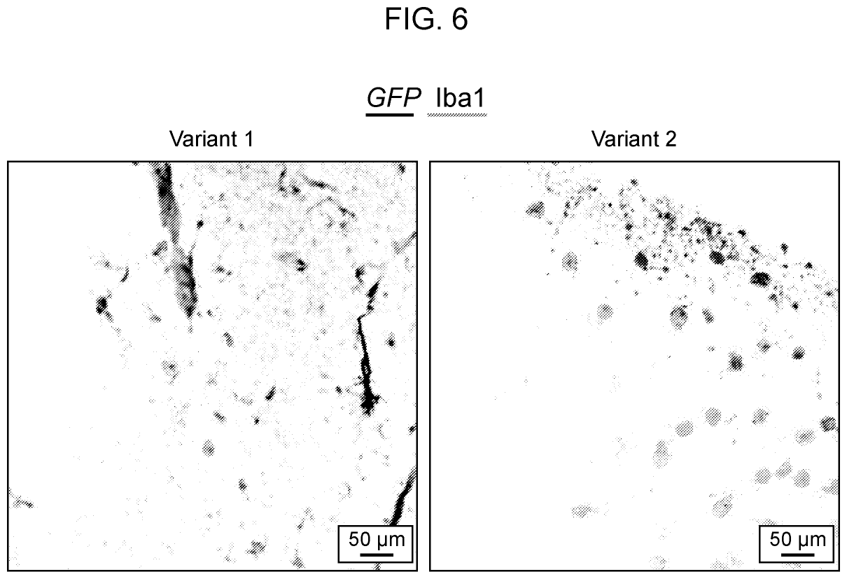

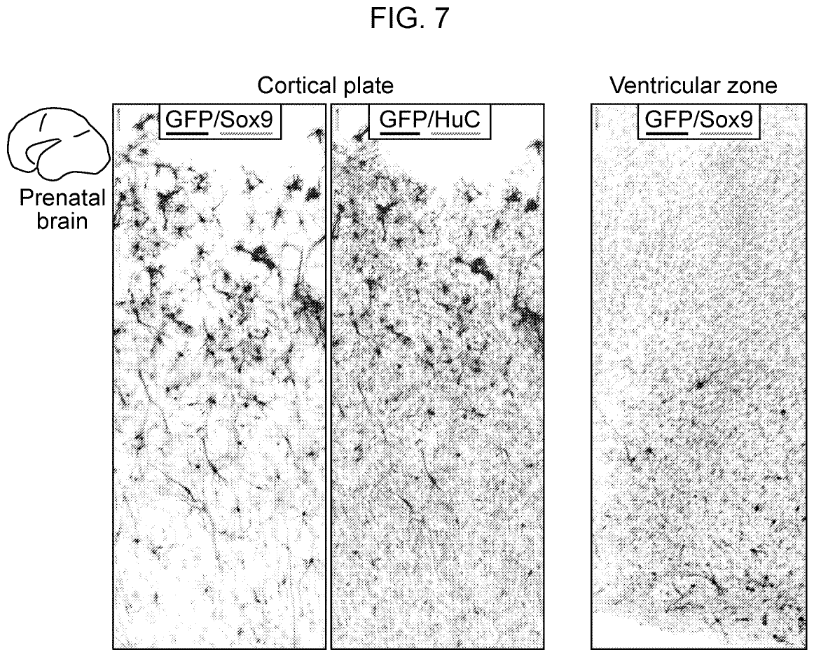

To validate if the evolved AAV variants can transduce neuronal/glial/microglial cells more efficiently and specifically, the selective cap gene within AAV genome were re-packaged into viral particles using GFP and administrated onto brain slices at MOI=1,000 in two separate rounds (i.e., different biological replicates). Seven days after infection, brain slices were fixed and immunostaining was performed against GFP and cell-specific markers on infected slices. Representative images also shown in FIGS. 3-4 and 6-7. Immunostaining showed high levels of glial infection across multiple regions of the primary brain tissue (FIG. 5).
7-amino acid insertion sequences for cell-specific AAVs (AAV5-based):

Glial-specific variants:
(SEQ ID NO: 42)
KVSNAAN,
(SEQ ID NO: 43)
VVKQRGD,
(SEQ ID NO: 12)
VTNVVRA,
(SEQ ID NO: 13)
PGPGNTI,
and
(SEQ ID NO: 14)
QRIVNEV.
Neuronal-specific variants:
(SEQ ID NO: 38)
KSSKNAT,
(SEQ ID NO: 40)
PKSRAGA,
(SEQ ID NO: 41)
RARGDGG,
(SEQ ID NO: 16)
RSSNYVV,
(SEQ ID NO: 17)
AAPIDGE,
(SEQ ID NO: 18)
IGPVAAD,
(SEQ ID NO: 19)
DGVEDAV,
(SEQ ID NO: 20)
GFTDDAT,
and
(SEQ ID NO: 21)
LEAVATV.
Microglia-specific variants:
(SEQ ID NO: 10)
KVTRGDT,
(SEQ ID NO: 44)
PADNVKA,
(SEQ ID NO: 22)
VEAVGGNVEAVGGN,
(SEQ ID NO: 23)
MSLLPYP,
(SEQ ID NO: 24)
YFAIYIF,
(SEQ ID NO: 25)
HLSDARP,
(SEQ ID NO: 39)
CHERRRV,
(SEQ ID NO: 26)
LIASFVQ,
(SEQ ID NO: 27)
AGGGGKA,
(SEQ ID NO: 28)
KGVQERA,
and
(SEQ ID NO: 29)
AVRINPG.

Additional highly-efficient AAV variants that infect primary human brain tissue were identified using the methods disclosed herein. These AAV variants included capsids encoded by the following sequences:

R1-4 (SCHEMA variant):
(SEQ ID NO: 31)
AATGTTCTCGTCACGTGGGCATGAATCTGATGCTGTTTCCCTGCAGACAATGC
GAGAGAATGAATCAGAATTCAAATATCTGCTTCACTCACGGACAGAAAGACTGTTTA
GAGTGCTTTCCCGTGTCAGAATCTCAACCCGTTTCTGTCGTCAAAAAGGCGTATCAG
AAACTGTGCTACATTCATCATATCATGGGAAAGGTGCCAGACGCTTGCACTGCCTGC
GATCTGGTCAATGTGGATTTGGATGACTGCATCTTTGAACAATAAATGATTTGCGAT
TTAAATCAGGTATGGCTGCCGATGGTTATCTTCCAGATTGGCTCGAGGACAACCTCT
CTGAGGGCATTCGCGAGTGGTGGGCGCTGAAACCTGGAACCCCGAAGCCCAAAGCC
AACCAGCAAAAGCAGGACGACGGCCGGGGTCTGGTGCTTCCTGGCTACAAGTACCT
CGGACCCTTCAACGGACTCGACAAGGGGGAGCCCGTCAACGCGGCGGACGCAGCGG
CCCTCGAGCACGACAAGGCCTACGACCAGCAGCTCAAAGCGGGTGACAATCCGTAC
CTGCGGTATAACCACGCCGACGCCGAGTTTCAGGAGCGTCTGCAAGAAGATACGTCT
TTTGGGGGCAACCTCGGGCGAGCAGTCTTCCAGGCCAAGAAGAGGGTTCTCGAACC
TTTTGGTCTGGTTGAGGAAGGTGCTAAGACGGCTCCTGGAAAGAAACGTCCGGTAG
AGCAGTCGCCACAAGAGCCAGACTCCTCCTCGGGCATTGGCAAGACAGGCCAGCAG
CCCGCTAAAAAGAGACTCAATTTTGGTCAGACTGGCGACTCAGAGTCAGTCCCCGAC
CCACAACCTCTCGGAGAACCTCCAGCAACCCCCGCTGCTGTGGGACCTACTACAATG
GCTTCAGGCGGTGGCGCACCAATGGCAGACAATAACGAAGGCGCCGACGGAGTGGG
TAATGCCTCAGGAAATTGGCATTGCGATTCCACATGGCTGGGCGACAGAGTCATCAC
CACCAGCACCCGAACATGGGCCTTGCCCACCTATAACAACCACCTCTACAAGCAAAT
CTCCAGTGCTTCAACGGGGGCCAGCAACGACAACCACTACTTCGGCTACAGCACCCC
CTGGGGGTATTTTGATTTCAACAGATTCCACTGCCATTTCTCACCACGTGACTGGCAG
CGACTCATCAACAACAATTGGGGATTCCGGCCCAAGAGACTCAACTTCAAGCTCTTC
AACATCCAAGTCAAGGAGGTCACGACGAATGATGGCGTCACGACCATCGCTAATAA
CCTTACCAGCACGGTTCAAGTCTTTACGGACTCGGAGTACCAGCTGCCGTACGTTCT
CGGCTCTGCCCACCAGGGCTGCCTGCCTCCGTTCCCGGCGGACGTGTTCATGATTCC
CCAGTACGGCTACCTAACACTCAACAACGGTAGTCAGGCCGTGGGACGCTCCTCCTT
CTACTGCCTGGAATACTTTCCTTCGCAGATGCTGAGAACCGGCAACAACTTCCAGTT
TACCTACACTTTTGAGGACGTTCCTTTCCACAGCAGCTACGCTCACAGCCAGAGTCT
GGACCGTCTCATGAATCCTCTCATCGACCAGTACCTGTATTACTTGAGCAGAACAAA
CACTCCAAGTGGAACCACCACGCAGTCAAGGCTTCAGTTTTCTCAGGCCGGAGCGAG
TGACATTCGGGACCAGTCTAGGAACTGGCTTCCTGGACCCTGTTACCGCCAGCAGCG
AGTATCAAAGACATCTGCGGATAACAACAACAGTGAATACTCGTGGACTGGAGCTA
CCAAGTACCACCTCAATGGCAGAGACTCTCTGGTGAATCCGGGCCCGGCCATGGCA
AGCCACAAGGACGATGAAGAAAAGTTTTTTCCTCAGAGCGGGGTTCTCATCTTTGGG
AAGCAAGGCTCAGAGAAAACAAATGTGGACATTGAAAAGGTCATGATTACAGACGA
AGAGGAAATCAGGACAACCAATCCCGTGGCTACGGAGCAGTATGGTTCTGTATCTA
CCAACCTCCAGAGAGGCAACAGACAAGCAGCTACCGCAGATGTCAACACACAAGGC
GTTCTTCCAGGCATGGTCTGGCAGGACAGAGACGTGTACCTTCAGGGGCCCATCTGG
GCAAAGATTCCACACACGGACGGACATTTTCACCCCTCTCCCCTCATGGGTGGATTC
GGACTTAAACACCCTCCTCCCCAGATTCTCATCAAGAACACCCCGGTACCTGCGAAT
CCTTCGACCACCTTCAACCAGTCAAAGCTGAACTCTTTCATCACCCAGTATTCTACTG
GCCAAGTCAGCGTGGAGATCGAGTGGGAGCTGCAGAAGGAAAACAGCAAGCGCTG
GAACCCGGAGATCCAGTACACTTCCAACTATTACAAGTCTAATAATGTTGAATTTGC
TGTTAATACTGAAGGTGTATATAGTGAACCCCGCCCCATTGGCACCAGATACCTGAC
TCGTAATCTGTAATTGCGGCCGCTTGTTAATCAATAAACCGTTTAATTCGTTTCAGTT
GAACTTTGGTCTCTGCGTATTTCTTTCTTATCTAGTTTCCATGCCTCGAGATAACTTCG
TATAATGTATGCTATACGAACGGTACTGT
A8 variant (SHUFFLE variant):
(SEQ ID NO: 32)
AACAATGTTCTCGTCACGTGGGCATGAATCTGATGCTGTTTCCCTGCAGACA
ATGCGAGAGAATGAATCAGAATTCAAATATCTGCTTCACTCACGGACAGAAAGACT
GTTTAGAGTGCTTTCCCGTGTCAGAATCTCAACCCGTTTCTGTCGTCAAAAAGGCGT
ATCAGAAACTGTGCTACATTCATCATATCATGGGAAAGGTGCCAGACGCTTGCACTG
CCTGCGATCTGGTCAATGTGGATTTGGATGACTGCATCTTTGAACAATAAATGATTT
AAATCAGGTATGGCTGCCGATGGTTATCTTCCAGATTGGCTCGAGGACACTCTCTCT
GAAGGAATAAGACAGTGGTGGAAGCTCAAACCTGGCCCACCACCACCAAAGCCCGC
AGAGCGGCATAAGGACGACAGCAGGGGTCTTGTGCTTCCTGGGTACAAGTACCTCG
GACCCTTCAACGGACTCGACAAGGGAGAGCCGGTCAACGAGGCAGACGCCGCGGCC
CTCGAGCACGACAAAGCCTACGACCGGCAGCTCGACAGCGGAGACAACCCGTACCT
CAAGTACAACCACGCCGACGCGGAGTTTCAGGAGCGCCTTAAAGAAGATACGTCTT
TTGGGGGCAACCTCGGACGAGCAGTCTTCCAGGCGAAAAAGAGGGTTCTTGAACCT
CTTGGTCTGGTTGAGGAAGCGGCTAAGACGGCTCCTGGAAAGAAGAGGCCTGTAGA
GCAGTCTCCTCAGGAACCGGACTCCTCCGCGGGTATTGGCAAATCGGGTGCACAGCC
CGCTAAAAAGAGACTCAATTTCGGTCAGACTGGCGACACAGAGTCAGTCCCAGACC
CTCAACCAATCGGAGAACCTCCCGCAGCCCCCTCAGGTGTGGGATCTCTTACAATGG
CTTCAGGTGGTGGCGCACCAGTGGCAGACAATAACGAAGGTGCCGATGGAGTGGGT
AGTTCCTCGGGAAATTGGCATTGCGATTCCCAATGGCTGGGGGACAGAGTCATCACC
ACCAGCACCCGAACCTGGGCCCTGCCCACCTACAACAATCACCTCTACAAGCAAATC
TCCAACAGCACATCTGGAGGATCTTCAAATGACAACGCCTACTTCGGCTACAGCACC
CCCTGGGGGTATTTTGACTTCAACAGATTCCACTGCCACTTCTCACCACGTGACTGGC
AGCGACTCATCAACAACAACTGGGGATTCCGGCCTAAGCGACTCAACTTCAAGCTCT
TCAACATTCAGGTCAAAGAGGTTACGGACAACAATGGAGTCAAGACCATCGCCAAT
AACCTTACCAGCACGGTCCAGGTCTTTACGGACTCGGAGTACCAGCTGCCGTACGTT
CTCGGCTCTGCCCACCAGGGCTGCCTGCCTCCGTTCCCGGCGGACGTGTTCATGATTC
CCCAGTACGGCTACCTAACACTCAACAACGGTAGTCAGGCCGTGGGACGCTCCTCCT
TCTACTGCCTGGAATACTTTCCTTCGCAGATGCTGAGAACCGGCAACAACTTCCAGT
TTACCTACACTTTTGAGGACGTTCCTTTCCACAGCAGCTACGCTCACAGCCAGAGTCT
GGACCGTCTCATGAATCCTCTCATCGACCAGTACCTGTATTACTTGAGCAGAACAAA
CACTCCAAGTGGAACCACCACGCAGTCAAGGCTTCAGTTTTCTCAGGCCGGAGCGAG
TGACATTCGGGACCAGTCTAGGAACTGGCTTCCTGGACCCTGTTACCGCCAGCAGCG
AGTATCAAAGACATCTGCGGATAACAACAACAGTGAATACTCGTGGACTGGAGCTA
CCAAGTACCACCTCAATGGCAGAGACTCTCTGGTGAATCCGGGCCCGGCCATGGCA
AGCCACAAGGACGATGAAGAAAAGTTTTTTCCTCAGAGCGGGGTTCTCATCTTTGGG
AAGCAAGGCTCAGAGAAAACAAATGTGGACATTGAAAAGGTCATGATTACAGACGA
AGAGGAAATCAGGACAACCAATCCCGTGGCTACGGAGCAGTATGGTTCTGTATCTA
CCAACCTCCAGAGAGGCAACAGACAAGCAGCTACCGCAGATGTCAACACACAAGGC
GTTCTTCCAGGCATGGTCTGGCAGGACAGAGACGTGTACCTGCAGGGTCCCATCTGG
GCCAAGATTCCTCACACGGACGGCAACTTCCACCCGTCTCCGCTGATGGGCGGCTTT
GGCCTGAAACATCCTCCGCCTCAGATCCTGATCAAGAACACGCCTGTACCTGCGGAT
CCTCCGACCACCTTCAACCAGTCAAAGCTGAACTCTTTCATCACCCAGTATTCTACTG
GCCAAGTCAGCGTGGAGATCGAGTGGGAGCTGCAGAAGGAAAACAGCAAGCGCTG
GAACCCGGAGATCCAGTACACTTCCAACTATTACAAGTCTAATAATGTTGAATTTGC
TGTTAATACTGAAGGTGTATATAGTGAACCCCGCCCCATTGGCACCAGATACCTGAC
TCGTAATCTGTAATTGCGGCCGCTTGTTAATCAATAAACCGTTTAATTCGTTTCAGTT
GAACTTTGGTCTCTGCGTATTTCTTTCTTATCTAGTTTCCATGCCTCGAGATAACTTCG
TATAATGTATGCTATACGAACGGTACTGTGGTCGTCATGCAA
A11 variant (SHUFFLE variant):
(SEQ ID NO: 33)
AACAATGTTCTCGTCACGTGGGCATGAATCTGATGCTGTTTCCCTGCAGACAATGCG
AGAGAATGAATCAGAATTCAAATATCTGCTTCACTCACGGACAGAAAGACTGTTTAG
AGTGCTTTCCCGTGTCAGAATCTCAACCCGTTTCTGTCGTCAAAAAGGCGTATCAGA
AACTGTGCTACATTCATCATATCATGGGAAAGGTGCCAGACGCTTGCACTGCCTGCG
ATCTGGTCAATGTGGATTTGGATGACTGCATCTTTGAACAATAAATGATTTGCGATTT
AAATCAGGTATGGCTGCCGATGGTTATCTTCCAGATTGGCTCGAGGACAACCTCTCT
GAGGGCATTCGCGAGTGGTGGGCGCTGAAACCTGGAGCCCCGAAGCCCAAAGCCAA
CCAGCAAAAGCAGGACGACGGCCGGGGTCTGGTGCTTCCTGGCTACAAGTACCTCG
GACCCGGCAACGGACTCGACAAGGGGGAGCCCGTCAACGCGGCGGACGCAGCGGC
CCTCGAGCACGACAAGGCCTACGACCAGCAGCTCAAAGCGGGTGACAATCCGTACC
TTCGGTATAACCACGCCGACGCCGAGTTTCAGGAGCGTCTGCAAGAAGATACGTCTT
TTGGGGGCAACCTCGGACGAGCAGTCTTCCAGGCCAAGAAAAGGGTTCTCGAACCT
TTTGGTCTGGTTGAGGAAGCGGCTAAGACGGCTCCTGGAAAGAAACGTCCGGTAGA
GCAGTCGCCACAAGAGCCAGACTCCTCCTCGGGCATCGGCAAGACAGGCCAGCAGC
CCGCTAAAAAGAGACTCAATTTTGGTCAGACTGGCGACTCAGAGTCAGTCCCCGACC
CACAACCTCTCGGAGAACCTCCAGCAACCCCCGCTGCTGTGGGACCTACTACAATGG
CTTCAGGCGGTGGCGCACCAATGGCAGACAATAACGAAGGCGCCGACGGAGTGGGT
AGTTCCTCGGGAAATTGGCATTGCGATTCCACATGGCTGGGCGACAGAGTCATCACC
ACCAGCACCCGCACCTGGGCCTTGCCCACCTATAACAACCACCTCTACAAGCAAATC
TCCAGTGCTTCAACGGGGGCCAGCAACGACAACCACTACTTCGGCTACAGCACCCCC
TGGGGGTATTTTGATTTCAACAGATTCCACTGCCATTTCTCACCACGTGACTGGCAGC
GACTCATCAACAACAACTGGGGATTCCGGCCCAAGAGACTCAACTTCAAACTCTTCA
ACATCCAAGTCAAGGAGGTCACGACGAATGATGGCGTCACAACCATCGCTAATAAC
CTTACCAGCACGGTTCAAGTCTTCTCGGACTCGGAGTACCAGTTGCCGTACGTCCTC
GGCTCTGCGCACCAGGGCTGCCTCCCTCCGTTCCCGGCGGACGTGTTCATGATTCCG
CAGTACGGCTACCTAACGCTCAACAATGGCAGCCAGGCAGTGGGACGGTCATCCTTT
TACTGCCTGGAATATTTCCCATCGCAGATGCTGAGAACCGGCAACAACTTCCAGTTT
ACTTACACCTTCGAGGACGTGCCTTTCCACAGCAGCTACGCGCACAGCCAGAGCCTG
GACCGGCTGATGAATCCTCTCATCGACCAGTACCTGTATTACCTGAACAGAACTCAG
AATCAGTCCGGAAGTGCCCAAAACAAGGACTTGCTGTTTAGCCGTGGGTCTCCAGCT
GGCATGTCTGTTCAGCCCAAAAACTGGCTACCTGGACCCTGTTATCGGCAGCAGCGC
GTTTCTAAAACAAAAACAGACAACAACAACAGCAATTTTACCTGGACTGGTGCTTCA
AAATATAACCTCAATGGGCGTGAATCCATCATCAACCCTGGCACTGCTATGGCCTCA
CACAAAGACGACGAAGACAAGTTCTTTCCCATGAGCGGTGTCATGATTTTTGGAAAA
GAGAGCGCCGGAGCTTCAAACACTGCATTGGACAATGTCATGATTACAGACGAAGA
GGAAATTAAAGCCACTAACCCTGTGGCCACCGAAAGATTTGGGACCGTGGCAGTCA
ATTTCCAGAGCAGCAGCACAGACCCTGCGACCGGAGATGTGCATGCTATGGGAGCA
TTACCTGGCATGGTGTGGCAAGATAGAGACGTGTACCTGCAGGGTCCCATTTGGGCC
AAAATTCCTCACACAGATGGACACTTTCACCCGTCTCCTCTCATGGGCGGCTTTGGA
CTTAAGCACCCGCCTCCTCAGATCCTCATCAAAAACACGCCTGTTCCTGCGAATCCT
CCGGCAGAGTTTTCGGCTACAAAGTTTGCTTCATTCATCACCCAGTATTCCACAGGA
CAAGTGAGCGTGGAGATTGAATGGGAGCTGCAGAAAGAAAACAGCAAACGCTGGA
ATCCCGAAGTGCAGTATACATCTAACTATGCAAAATCTGCCAACGTTGATTTCACTG
TGGACAACAATGGACTTTATACTGAGCCTCGCCCCATTGGCACCCGTTACCTCACCC
GTCCCCTGTAATTGCGGCCGCTTGTTAATCAATAAACCGTTTAATTCGTTTCAGTTGA
ACTTTGGTCTCTGCGTATTTCTTTCTTATCTAGTTTCCATGCCTCGAGATAACTTCGTA
TAATGTATGCTATACGAACGGTACTGT

The capsid proteins encoded by the above listed nucleic acid sequences are as follows:

Capsid encoded by R1-4 (SCHEMA variant):
(SEQ ID NO: 34)
MAADGYLPDWLEDNLSEGIREWWALKPGTPKPKANQQKQDDGRGLVLPGYKY
LGPFNGLDKGEPVNAADAAALEHDKAYDQQLKAGDNPYLRYNHADAEFQERLQEDTSFGGNL
GRAVFQAKKRVLEPFGLVEEGAKTAPGKKRPVEQSPQEPDSSSGIGKTGQQPAKKRLNFGQTGD
SESVPDPQPLGEPPATPAAVGPTTMASGGGAPMADNNEGADGVGNASGNWHCDSTWLGDRVI
TTSTRTWALPTYNNHLYKQISSASTGASNDNHYFGYSTPWGYFDFNRFHCHFSPRDWQRLINNN
WGFRPKRLNFKLFNIQVKEVTTNDGVTTIANNLTSTVQVFTDSEYQLPYVLGSAHQGCLPPFPA
DVFMIPQYGYLTLNNGSQAVGRSSFYCLEYFPSQMLRTGNNFQFTYTFEDVPFHSSYAHSQSLD
RLMNPLIDQYLYYLSRTNTPSGTTTQSRLQFSQAGASDIRDQSRNWLPGPCYRQQRVSKTSADN
NNSEYSWTGATKYHLNGRDSLVNPGPAMASHKDDEEKFFPQSGVLIFGKQGSEKTNVDIEKVMI
TDEEEIRTTNPVATEQYGSVSTNLQRGNRQAATADVNTQGVLPGMVWQDRDVYLQGPIWAKIP
HTDGHFHPSPLMGGFGLKHPPPQILIKNTPVPANPSTTFNQSKLNSFITQYSTGQVSVEIEWELQK
ENSKRWNPEIQYTSNYYKSNNVEFAVNTEGVYSEPRPIGTRYLTRNL
Capsid encoded by A8 variant (SHUFFLE variant):
(SEQ ID NO: 35)
MAADGYLPDWLEDTLSEGIRQWWKLKPGPPPPKPAERHKDDSRGLVLPGYKYLGPFNGLDKGE
PVNEADAAALEHDKAYDRQLDSGDNPYLKYNHADAEFQERLKEDTSFGGNLGRAVFQAKKRV
LEPLGLVEEAAKTAPGKKRPVEQSPQEPDSSAGIGKSGAQPAKKRLNFGQTGDTESVPDPQPIGE
PPAAPSGVGSLTMASGGGAPVADNNEGADGVGSSSGNWHCDSQWLGDRVITTSTRTWALPTY
NNHLYKQISNSTSGGSSNDNAYFGYSTPWGYFDFNRFHCHFSPRDWQRLINNNWGFRPKRLNF
KLFNIQVKEVTDNNGVKTIANNLTSTVQVFTDSEYQLPYVLGSAHQGCLPPFPADVFMIPQYGY
LTLNNGSQAVGRSSFYCLEYFPSQMLRTGNNFQFTYTFEDVPFHSSYAHSQSLDRLMNPLIDQYL
YYLSRTNTPSGTTTQSRLQFSQAGASDIRDQSRNWLPGPCYRQQRVSKTSADNNNSEYSWTGAT
KYHLNGRDSLVNPGPAMASHKDDEEKFFPQSGVLIFGKQGSEKTNVDIEKVMITDEEEIRTTNPV
ATEQYGSVSTNLQRGNRQAATADVNTQGVLPGMVWQDRDVYLQGPIWAKIPHTDGNFHPSPL
MGGFGLKHPPPQILIKNTPVPADPPTTFNQSKLNSFITQYSTGQVSVEIEWELQKENSKRWNPEIQ
YTSNYYKSNNVEFAVNTEGVYSEPRPIGTRYLTRNL
Capsid encoded by A11 variant (SHUFFLE variant):
(SEQ ID NO: 36)
MAADGYLPDWLEDNLSEGIREWWALKPGAPKPKANQQKQDDGRGLVLPGYKYLGPGNGLDK
GEPVNAADAAALEHDKAYDQQLKAGDNPYLRYNHADAEFQERLQEDTSFGGNLGRAVFQAK
KRVLEPFGLVEEAAKTAPGKKRPVEQSPQEPDSSSGIGKTGQQPAKKRLNFGQTGDSESVPDPQP
LGEPPATPAAVGPTTMASGGGAPMADNNEGADGVGSSSGNWHCDSTWLGDRVITTSTRTWAL
PTYNNHLYKQISSASTGASNDNHYFGYSTPWGYFDFNRFHCHFSPRDWQRLINNNWGFRPKRL
NFKLFNIQVKEVTTNDGVTTIANNLTSTVQVFSDSEYQLPYVLGSAHQGCLPPFPADVFMIPQYG
YLTLNNGSQAVGRSSFYCLEYFPSQMLRTGNNFQFTYTFEDVPFHSSYAHSQSLDRLMNPLIDQ
YLYYLNRTQNQSGSAQNKDLLFSRGSPAGMSVQPKNWLPGPCYRQQRVSKTKTDNNNSNFTW
TGASKYNLNGRESIINPGTAMASHKDDEDKFFPMSGVMIFGKESAGASNTALDNVMITDEEEIK
ATNPVATERFGTVAVNFQSSSTDPATGDVHAMGALPGMVWQDRDVYLQGPIWAKIPHTDGHF
HPSPLMGGFGLKHPPPQILIKNTPVPANPPAEFSATKFASFITQYSTGQVSVEIEWELQKENSKRW
NPEVQYTSNYAKSANVDFTVDNNGLYTEPRPIGTRYLTRPL

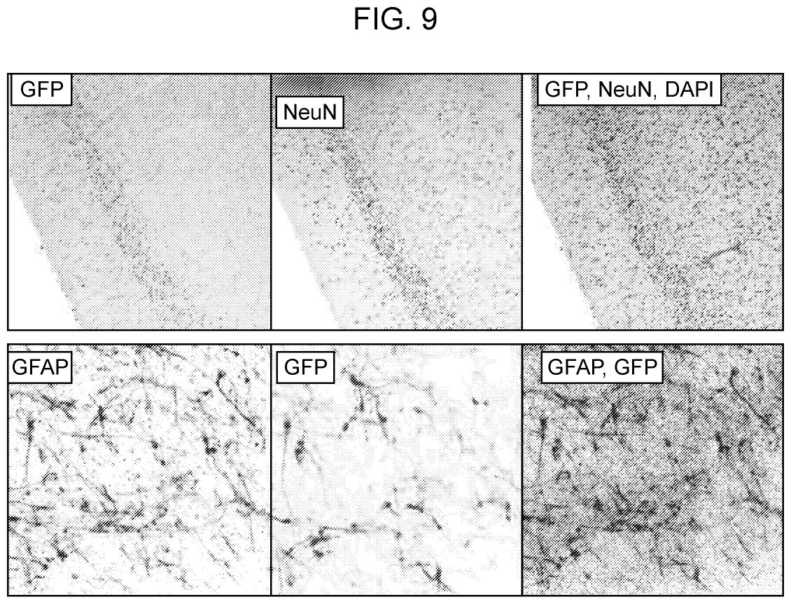

FIG. 9. AAV libraries were selected for the capacity to infect primary human brain tissue. Following library infection, different cell populations were sorted from dissociated tissue, and recovered AAV genomes were subjected to next-generation sequencing. Top variants recovered from the neuronal fraction or astrocytic fraction were packaged with GFP and then individually applied to primary brain slices. The N5 variant (GFTDDAT (SEQ ID NO:20)) infects neurons (in human cortical slices) selectively and very efficiently at a MOI of 1000 (top row), and the G4 variant (VVKQRGD; SEQ ID NO:43) infects astrocytes neurons (in human cortical slices) selectively and efficiently (bottom row).

In conclusion, a list of highly-efficient AAV variants has developed, using vector engineering with a machine-learning designed library, that overcome current challenges for cell-specific delivery to the human CNS. The AAV vectors described here were all developed in the context of primary human brain tissue, which is an important preclinical model for human biology and diseases. Therefore, they were endowed with higher efficiency and the capacity to target critical and rare population cell types in the human brain more efficiently compared to other AAV vectors.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A recombinant adeno-associated virus (rAAV) comprising:
(a) a variant AAV capsid protein, wherein the variant AAV capsid protein comprises an insertion of a heterologous peptide comprising the amino acid sequence:
```
  1)
(SEQ ID NO: 42)
KVSNAAN;
  2)
(SEQ ID NO: 43)
VVKQRGD;
  3)
(SEQ ID NO: 12)
VTNVVRA;
  4)
(SEQ ID NO: 13)
PGPGNTI;
  5)
(SEQ ID NO: 14
QRIVNEV;
  6)
(SEQ ID NO: 38)
KSSKNAT;
  7)
(SEQ ID NO: 40)
PKSRAGA;
  8)
(SEQ ID NO: 41)
RARGDGG;
  9)
(SEQ ID NO: 16)
RSSNYVV;
10)
(SEQ ID NO: 17)
AAPIDGE;
11)
(SEQ ID NO: 18)
IGPVAAD;
12)
(SEQ ID NO: 19)
DGVEDAV;
13)
(SEQ ID NO: 20)
GFTDDAT;
14)
(SEQ ID NO: 21)
LEAVATV;
15)
(SEQ ID NO: 10)
KVTRGDT;
16)
(SEQ ID NO: 44)
PADNVKA;
17)
(SEQ ID NO: 45)
RDGGTKA;
18)
(SEQ ID NO: 22)
VEAVGGNVEAVGGN;
19)
(SEQ ID NO: 23)
MSLLPYP;
20)
(SEQ ID NO: 24)
YFAIYIF;
21)
(SEQ ID NO: 25)
HLSDARP;
22)
(SEQ ID NO: 39)
CHERRRV;
23)
(SEQ ID NO: 26)
LIASFVQ;
24)
(SEQ ID NO: 27)
AGGGGKA;
25)
(SEQ ID NO: 28)
KGVQERA;
or
26)
(SEQ ID NO: 29)
AVRINPG;
```
and
(b) a heterologous nucleic acid comprising a nucleotide sequence encoding a gene product.

2. The rAAV of claim 1, wherein the rAAV comprises the heterologous peptide comprising the amino acid sequence:
```
  1)
(SEQ ID NO: 42)
KVSNAAN;
  2)
(SEQ ID NO: 43)
VVKQRGD;
  3)
(SEQ ID NO: 12)
VTNVVRA;
  4)
(SEQ ID NO: 13)
PGPGNTI;
or
  5)
(SEQ ID NO: 14
QRIVNEV,
```
and wherein the rAAV exhibits increased infectivity of a glial cell compared to the infectivity of the glial cell by a control rAAV comprising the corresponding parental AAV capsid protein.

3. (canceled)

4. The rAAV of claim 1, wherein the rAAV comprises the heterologous peptide comprising the amino acid sequence:
```
  6)
(SEQ ID NO: 38)
KSSKNAT;
  7)
(SEQ ID NO: 40)
PKSRAGA;
  8)
(SEQ ID NO: 41)
RARGDGG;
  9)
(SEQ ID NO: 16)
RSSNYVV;
10)
(SEQ ID NO: 17)
AAPIDGE;
11)
(SEQ ID NO: 18)
IGPVAAD;
12)
(SEQ ID NO: 19)
DGVEDAV;
13)
(SEQ ID NO: 20)
GFTDDAT;
or
14)
(SEQ ID NO: 21)
LEAVATV,
```
and wherein the rAAV exhibits increased infectivity of a neuronal cell compared to the infectivity of the neuronal cell by a control rAAV comprising the corresponding parental AAV capsid protein.

5. (canceled)

6. The rAAV of claim 1, wherein the rAAV comprises the heterologous peptide comprising the amino acid sequence:
15)
(SEQ ID NO: 10)
KVTRGDT;
16)
(SEQ ID NO: 44)
PADNVKA;
17)
(SEQ ID NO: 45)
RDGGTKA;
18)
(SEQ ID NO: 22)
VEAVGGNVEAVGGN;
19)
(SEQ ID NO: 23)
MSLLPYP;
20)
(SEQ ID NO: 24)
YFAIYIF;
21)
(SEQ ID NO: 25)
HLSDARP;
22)
(SEQ ID NO: 39)
CHERRRV;
23)
(SEQ ID NO: 26)
LIASFVQ;
24)
(SEQ ID NO: 27)
AGGGGKA;
25)
(SEQ ID NO: 28)
KGVQERA;
or
26)
(SEQ ID NO: 29)
AVRINPG,
and wherein the rAAV exhibits increased infectivity of a microglial cell compared to the infectivity of the microglial cell by a control rAAV comprising the corresponding parental AAV capsid protein.

7. (canceled)

8. The rAAV of claim 1, wherein the heterologous peptide is inserted at an insertion site between amino acids corresponding to amino acids 575-577 of AAV5, or the corresponding position in the capsid protein of another AAV serotype.

9. The rAAV of claim 1, wherein the heterologous peptide is inserted at an insertion site between amino acids corresponding to amino acids 575-576 of AAV5, or the corresponding position in the capsid protein of another AAV serotype.

10-12. (canceled)

13. The rAAV of claim 1, wherein the gene product is a polypeptide and the polypeptide is an RNA-guided endonuclease selected from a type II CRISPR/Cas polypeptide, a type V CRISPR/Cas polypeptide, and a type VI CRISPR/Cas polypeptide.

14-15. (canceled)

16. A pharmaceutical composition comprising:
a) a recombinant adeno-associated virus of claim 1; and
b) a pharmaceutically acceptable excipient.

17. A method of delivering a gene product to a central nervous system cell in a subject, the method comprising administering to the subject an adeno-associated virus (rAAV) according to claim 1.

18-19. (canceled)

20. A method of treating a central nervous system disease, the method comprising administering to a subject in need thereof an effective amount of an adeno-associated virus (rAAV) according to claim 1.

21-22. (canceled)

23. An isolated nucleic acid comprising a nucleotide sequence that encodes a variant adeno-associated virus (AAV) capsid protein, wherein the variant AAV capsid protein comprises an insertion of a heterologous peptide comprising the amino acid sequence:
```
  1)
(SEQ ID NO: 42)
KVSNAAN;
  2)
(SEQ ID NO: 43)
VVKQRGD;
  3)
(SEQ ID NO: 12)
VTNVVRA;
  4)
(SEQ ID NO: 13)
PGPGNTI;
  5)
(SEQ ID NO: 14
QRIVNEV;
  6)
(SEQ ID NO: 38)
KSSKNAT;
  7)
(SEQ ID NO: 40)
PKSRAGA;
  8)
(SEQ ID NO: 41)
RARGDGG;
  9)
(SEQ ID NO: 16)
RSSNYVV;
10)
(SEQ ID NO: 17)
AAPIDGE;
11)
(SEQ ID NO: 18)
IGPVAAD;
12)
(SEQ ID NO: 19)
DGVEDAV;
13)
(SEQ ID NO: 20)
GFTDDAT;
14)
(SEQ ID NO: 21)
LEAVATV;
15)
(SEQ ID NO: 10)
KVTRGDT;
16)
(SEQ ID NO: 44)
PADNVKA;
17)
(SEQ ID NO: 45)
RDGGTKA;
18)
(SEQ ID NO: 22)
VEAVGGNVEAVGGN;
19)
(SEQ ID NO: 23)
MSLLPYP;
20)
(SEQ ID NO: 24)
YFAIYIF;
21)
(SEQ ID NO: 25)
HLSDARP;
22)
(SEQ ID NO: 39)
CHERRRV;
23)
(SEQ ID NO: 26)
LIASFVQ;
24)
(SEQ ID NO: 27)
AGGGGKA;
25)
(SEQ ID NO: 28)
KGVQERA;
or
26)
(SEQ ID NO: 29)
AVRINPG.
```

24-32. (canceled)

33. An isolated, genetically modified host cell comprising the nucleic acid of any one of claim 23 claims 23-32.

34. A variant adeno-associated virus (AAV) capsid protein encoded by the isolated nucleic acid of claim 23.

35. A host cell producing an AAV of claim 1.

36-44. (canceled)

45. A recombinant adeno-associated virus (rAAV) comprising:
(a) a variant AAV capsid protein comprising an amino acid sequence having at least 95% identity to the amino acid sequence:
(SEQ ID NO: 34)
MAADGYLPDWLEDNLSEGIREWWALKPGTPKPKANQQKQDDGRGLVLPGYKY
LGPFNGLDKGEPVNAADAAALEHDKAYDQQLKAGDNPYLRYNHADAEFQERLQEDTS
FGGNLGRAVFQAKKRVLEPFGLVEEGAKTAPGKKRPVEQSPQEPDSSSGIGKTGQQPAK
KRLNFGQTGDSESVPDPQPLGEPPATPAAVGPTTMASGGGAPMADNNEGADGVGNAS
GNWHCDSTWLGDRVITTSTRTWALPTYNNHLYKQISSASTGASNDNHYFGYSTPWGYF
DFNRFHCHFSPRDWQRLINNNWGFRPKRLNFKLFNIQVKEVTTNDGVTTIANNLTSTVQ
VFTDSEYQLPYVLGSAHQGCLPPFPADVFMIPQYGYLTLNNGSQAVGRSSFYCLEYFPS
QMLRTGNNFQFTYTFEDVPFHSSYAHSQSLDRLMNPLIDQYLYYLSRTNTPSGTTTQSRL
QFSQAGASDIRDQSRNWLPGPCYRQQRVSKTSADNNNSEYSWTGATKYHLNGRDSLV
NPGPAMASHKDDEEKFFPQSGVLIFGKQGSEKTNVDIEKVMITDEEEIRTTNPVATEQYG
SVSTNLQRGNRQAATADVNTQGVLPGMVWQDRDVYLQGPIWAKIPHTDGHFHPSPLM
GGFGLKHPPPQILIKNTPVPANPSTTFNQSKLNSFITQYSTGQVSVEIEWELQKENSKRWN
PEIQYTSNYYKSNNVEFAVNTEGVYSEPRPIGTRYLTRNL;
or
(SEQ ID NO: 35)
MAADGYLPDWLEDTLSEGIRQWWKLKPGPPPPKPAERHKDDSRGLVLPGYKYL
GPFNGLDKGEPVNEADAAALEHDKAYDRQLDSGDNPYLKYNHADAEFQERLKEDTSF
GGNLGRAVFQAKKRVLEPLGLVEEAAKTAPGKKRPVEQSPQEPDSSAGIGKSGAQPAK
KRLNFGQTGDTESVPDPQPIGEPPAAPSGVGSLTMASGGGAPVADNNEGADGVGSSSGN
WHCDSQWLGDRVITTSTRTWALPTYNNHLYKQISNSTSGGSSNDNAYFGYSTPWGYFD
FNRFHCHFSPRDWQRLINNNWGFRPKRLNFKLFNIQVKEVTDNNGVKTIANNLTSTVQV
FTDSEYQLPYVLGSAHQGCLPPFPADVFMIPQYGYLTLNNGSQAVGRSSFYCLEYFPSQ
MLRTGNNFQFTYTFEDVPFHSSYAHSQSLDRLMNPLIDQYLYYLSRTNTPSGTTTQSRLQ
FSQAGASDIRDQSRNWLPGPCYRQQRVSKTSADNNNSEYSWTGATKYHLNGRDSLVNP
GPAMASHKDDEEKFFPQSGVLIFGKQGSEKTNVDIEKVMITDEEEIRTTNPVATEQYGSV
STNLQRGNRQAATADVNTQGVLPGMVWQDRDVYLQGPIWAKIPHTDGNFHPSPLMGG
FGLKHPPPQILIKNTPVPADPPTTFNQSKLNSFITQYSTGQVSVEIEWELQKENSKRWNPE
IQYTSNYYKSNNVEFAVNTEGVYSEPRPIGTRYLTRNL;
or
(SEQ ID NO: 36)
MAADGYLPDWLEDNLSEGIREWWALKPGAPKPKANQQKQDDGRGLVLPGYKY
LGPGNGLDKGEPVNAADAAALEHDKAYDQQLKAGDNPYLRYNHADAEFQERLQEDTS
FGGNLGRAVFQAKKRVLEPFGLVEEAAKTAPGKKRPVEQSPQEPDSSSGIGKTGQQPAK
KRLNFGQTGDSESVPDPQPLGEPPATPAAVGPTTMASGGGAPMADNNEGADGVGSSSG
NWHCDSTWLGDRVITTSTRTWALPTYNNHLYKQISSASTGASNDNHYFGYSTPWGYFD
FNRFHCHFSPRDWQRLINNNWGFRPKRLNFKLFNIQVKEVTTNDGVTTIANNLTSTVQV
FSDSEYQLPYVLGSAHQGCLPPFPADVFMIPQYGYLTLNNGSQAVGRSSFYCLEYFPSQ
MLRTGNNFQFTYTFEDVPFHSSYAHSQSLDRLMNPLIDQYLYYLNRTQNQSGSAQNKD
LLFSRGSPAGMSVQPKNWLPGPCYRQQRVSKTKTDNNNSNFTWTGASKYNLNGRESII
NPGTAMASHKDDEDKFFPMSGVMIFGKESAGASNTALDNVMITDEEEIKATNPVATERF
GTVAVNFQSSSTDPATGDVHAMGALPGMVWQDRDVYLQGPIWAKIPHTDGHFHPSPL
MGGFGLKHPPPQILIKNTPVPANPPAEFSATKFASFITQYSTGQVSVEIEWELQKENSKR
WNPEVQYTSNYAKSANVDFTVDNNGLYTEPRPIGTRYLTRPL;
and
(b) a heterologous nucleic acid comprising a nucleotide sequence encoding a gene product.

46-54. (canceled)

55. A method of delivering a gene product to a central nervous system cell in a subject or treating a central nervous system disease in a subject, the method comprising administering to the subject an adeno-associated virus (rAAV) according to claim 45.

56-58. (canceled)

59. An isolated nucleic acid comprising a nucleotide sequence that encodes a variant adeno-associated virus (AAV) capsid protein comprising an amino acid sequence as set forth in claim 45.

60. An isolated, genetically modified host cell comprising the nucleic acid of claim 59.

61. A variant adeno-associated virus (AAV) capsid protein encoded by the isolated nucleic acid of claim 59.

62. A host cell producing an AAV of claim 45.
